(19) United States
(12) Patent Application Publication
(10) Pub. No.: US 2026/0055128 A1
(43) Pub. Date: Feb. 26, 2026

(54) ORGANONICKEL INTERMEDIATES USEFUL FOR CARBOXYLATION OF SUBSTRATES WITH CARBON DIOXIDE

(71) Applicants: Alliance for Sustainable Energy, LLC, Golden, CO (US); Brookhaven Science Associates, LLC, Upton, NY (US); The Board of Trustees of the Leland Stanford Junior University, Stanford, CA (US)

(72) Inventors: Max KUDISCH, Wheat Ridge, CO (US); Lakshmy Kannadi VALLOLI, Upton, NY (US); Reagan Xie-Mei HOOPER, Menlo Park, CA (US)

(21) Appl. No.: 19/308,627

(22) Filed: Aug. 25, 2025

Related U.S. Application Data
(60) Provisional application No. 63/686,695, filed on Aug. 23, 2024.

(51) Int. Cl. C07F 15/04; B01J 31/18; B01J 35/39; C07C 51/15; C07D 307/24; C07D 309/08; C07D 319/12

(52) U.S. Cl. CPC C07F 15/04; B01J 31/1815; B01J 35/39; C07C 51/15; C07D 307/24; C07D 309/08; C07D 319/12; B01J 2231/42; B01J 2531/004; B01J 2531/847; C07C 2601/08

(57) ABSTRACT

The present disclosure relates to a compound having the structure

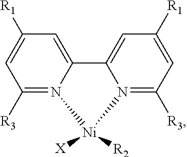

where R₁ includes at least one of a methyl group, a halogen, a hydrogen, or a combination thereof, R₂ includes at least one of a benzyl group, an ether, or a combination thereof, R₃ includes at least one of hydrogen, a methyl group, or a combination thereof, and X includes a halogen.

### CROSS-REFERENCE TO RELATED APPLICATIONS

This application claims priority from U.S. Provisional Patent Application No. 63/686,695 filed on Aug. 23, 2024 and its associated appendix, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

The increasing threats posed by global warming and climate change highlight the need to reduce reliance on fossil-based resources and transition toward sustainable alternatives. Biomass represents a renewable and widely available feedstock with significant potential for the production of fuels, chemicals, and materials. However, conventional biomass conversion processes sometimes suffer from inefficiencies, high costs, and limited scalability, which can restrict their economic viability. Accordingly, there remains a need for improved methods and materials that enable the efficient, cost-effective, and environmentally sustainable manufacture of useful products from biomass.

### SUMMARY

An aspect of the present disclosure is a compound having the structure

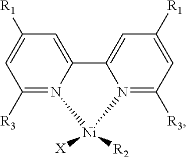

where R₁ includes at least one of a methyl group, a halogen, a hydrogen, or a combination thereof, R₂ includes at least one of a benzyl group, an ether, or a combination thereof, R₃ includes at least one of hydrogen, a methyl group, or a combination thereof, and X includes a halogen. In some embodiments of the present disclosure, R₂ may include an alkyl group linking R₂ to the nickel atom. In some embodiments of the present disclosure, the compound may further include an alkyl linking group positioned between the nickel atom and R₂. In some embodiments of the present disclosure, the alkyl group positioned between the nickel atom and R₂ may be a —CH₂— group. In some embodiments of the present disclosure, R₁ may include at least one of —C(CH₃)₃, —CF₃, H, or a combination thereof. In some embodiments of the present disclosure, X may include at least one iodine, chlorine, bromine, or a combination thereof.

In some embodiments of the present disclosure, the benzyl group may include at least one of

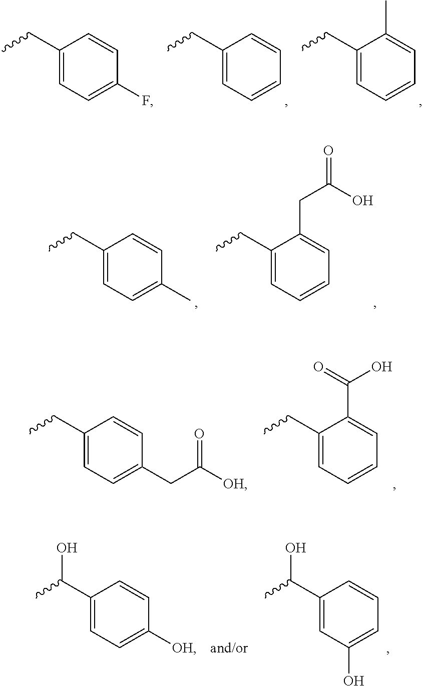

where [symbol] is a covalent bond between R₂ and Ni.

In some embodiments of the present disclosure, the ether may be a linear molecule. In some embodiments of the present disclosure, the linear molecule may include at least one of

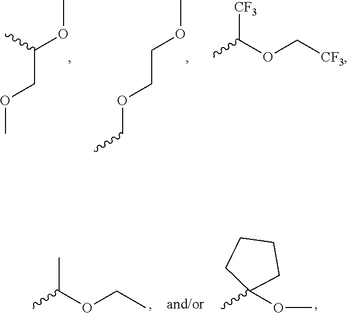

where [symbol] is a covalent bond between R₂ and Ni.

In some embodiments of the present disclosure, the ether may be a cyclic molecule. In some embodiments of the present disclosure, the cyclic molecule include at least one of

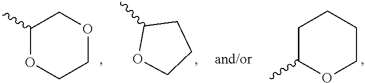

where [symbol] is a covalent bond between R₂ and Ni.

An aspect of the present disclosure is a method that includes reacting CO₂ and a nickel pre-catalyst with a reactant that includes at least one of a benzyl group, an ether, or a combination thereof to form a carboxylic acid, where the nickel pre-catalyst has the structure

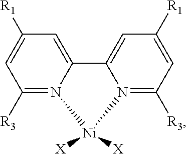

R₁ includes at least one of a methyl group, a halogen, a hydrogen, or a combination thereof, R₃ includes at least one of hydrogen, a methyl group, or a combination thereof, and X includes a halogen. In some embodiments of the present disclosure, the reacting may further include irradiating the nickel pre-catalyst with light having a wavelength between 300 nm and 800 nm or between 350 nm and 500 nm.

In some embodiments of the present disclosure, the reacting further includes a photosensitizer. In some embodiments of the present disclosure, the reacting further includes a base. In some embodiments of the present disclosure, the reacting further includes a sacrificial reductant.

In some embodiments of the present disclosure, the reacting further includes forming an intermediate compound prior to forming the carboxylic acid, and the intermediate compound has the structure

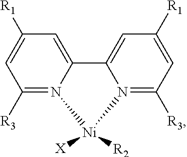

and R₂ includes at least one of the benzyl group, the ether, or a combination thereof.

In some embodiments of the present disclosure, the carboxylic acid may include at least one of

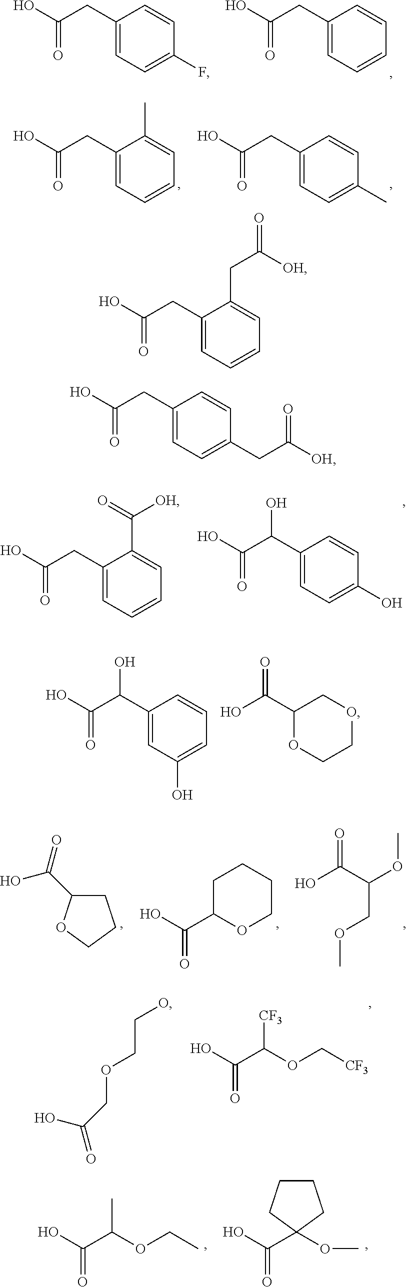

or a combination thereof.

In some embodiments of the present disclosure, the reactant may include at least one of

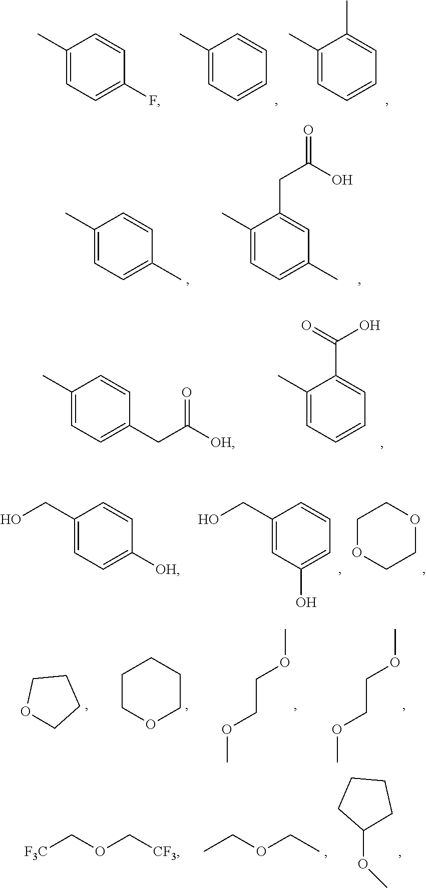

or a combination thereof.

In some embodiments of the present disclosure, the method may further include, prior to reacting CO₂ and the nickel pre-catalyst with the reactant, reacting a ligand with a nickel complex to form the nickel pre-catalyst, where the ligand has the structure

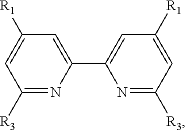

and the nickel complex has the structure

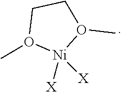

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates a mechanism for the reactions of a ligand, a nickel pre-catalyst, a reactant, and CO₂ in the presence of light to produce useful chemicals, according to some embodiments of the present disclosure.

FIG. 2 illustrates a method for converting biomass-derived reactants to carboxylic acids, according to some embodiments of the present disclosure.

FIG. 3A illustrates ultra-violet visible (UV-vis) spectroscopy data obtained for Experimental Compounds #1a and 1b (see Scheme 16 and Table 1), according to some embodiments of the present disclosure.

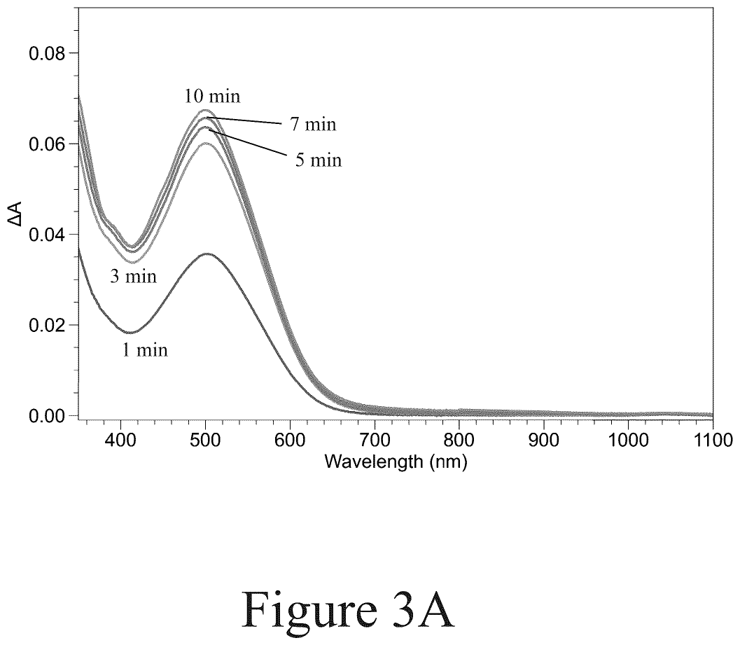

FIG. 3B illustrates nuclear magnetic resonance (NMR) data obtained for Experimental Compounds #1a and 1b (see Scheme 16 and Table 1), according to some embodiments of the present disclosure.

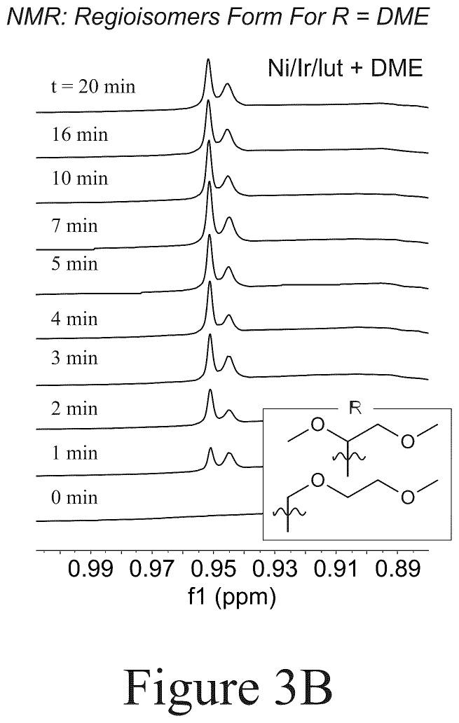

FIG. 4 illustrates UV-vis spectroscopy data obtained for Experimental Compound #2 (see Scheme 16 and Table 1), according to some embodiments of the present disclosure.

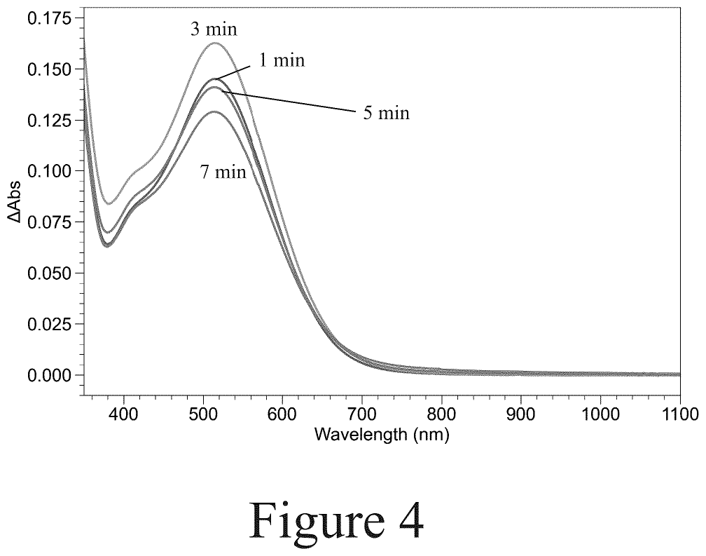

FIG. 5 illustrates UV-vis spectroscopy data obtained for Experimental Compound #3 (see Scheme 16 and Table 1), according to some embodiments of the present disclosure.

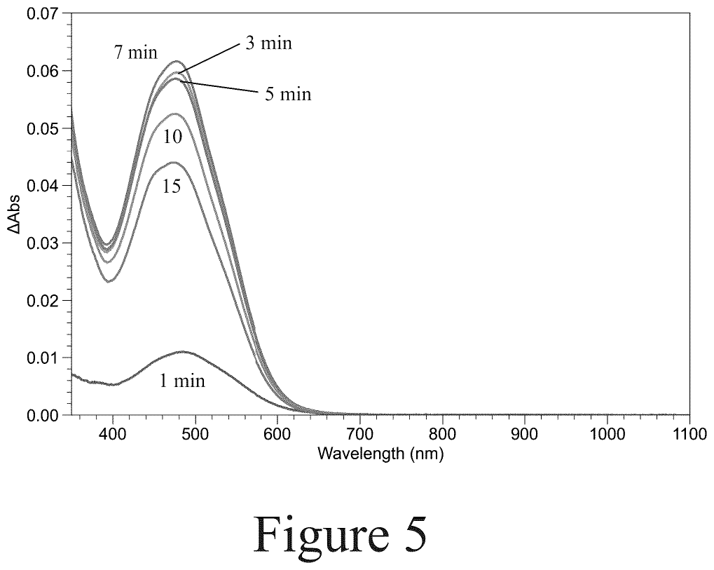

FIG. 6A illustrates NMR data obtained for Experimental Compound #4 (see Scheme 16 and Table 1), according to some embodiments of the present disclosure.

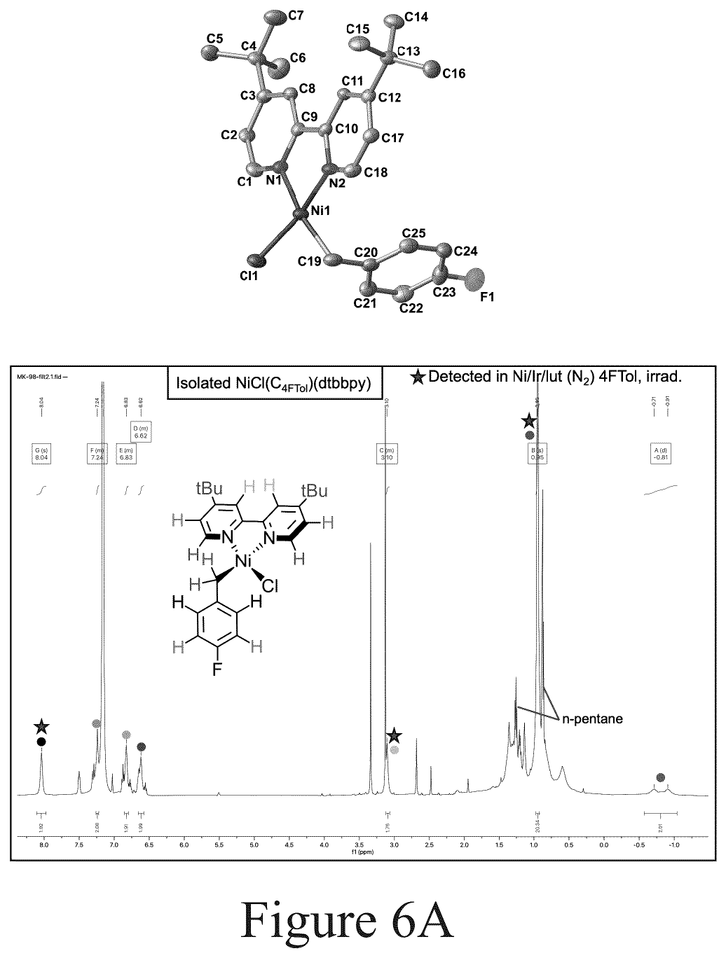

FIG. 6B illustrates UV-vis spectroscopy data obtained for Experimental Compound #4 (see Scheme 16 and Table 1), according to some embodiments of the present disclosure.

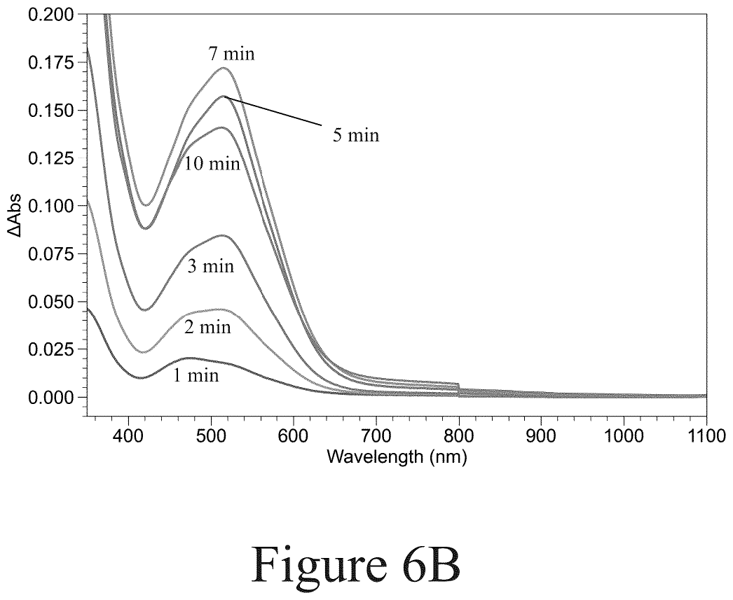

FIG. 6C illustrates X-ray diffraction (XRD) data obtained for Experimental Compound #4 (see Scheme 16 and Table 1), according to some embodiments of the present disclosure.

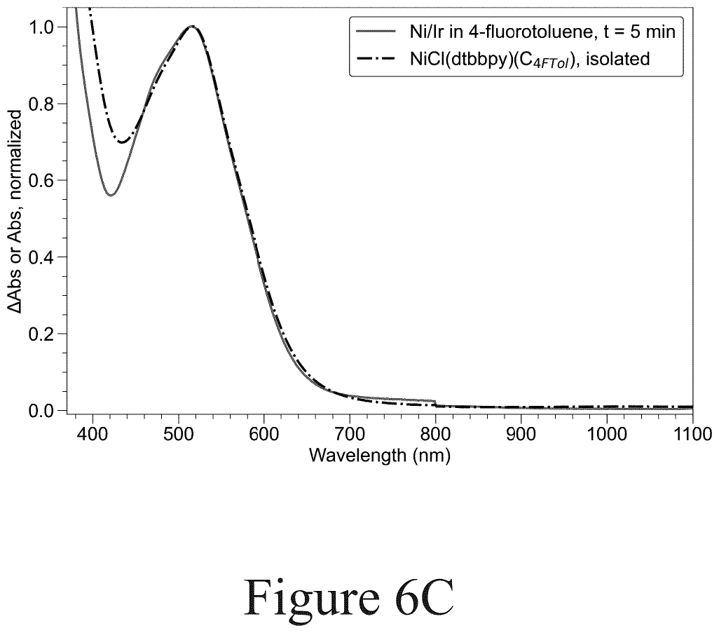

FIG. 6D illustrates ¹⁹F NMR of crude and dried reaction mixture showing a signal at −116.97 ppm that aligns well with the commercially purchased product, 4-fluorophenylacetic acid (4-FPhAc, −116.95), according to some embodiments of the present disclosure.

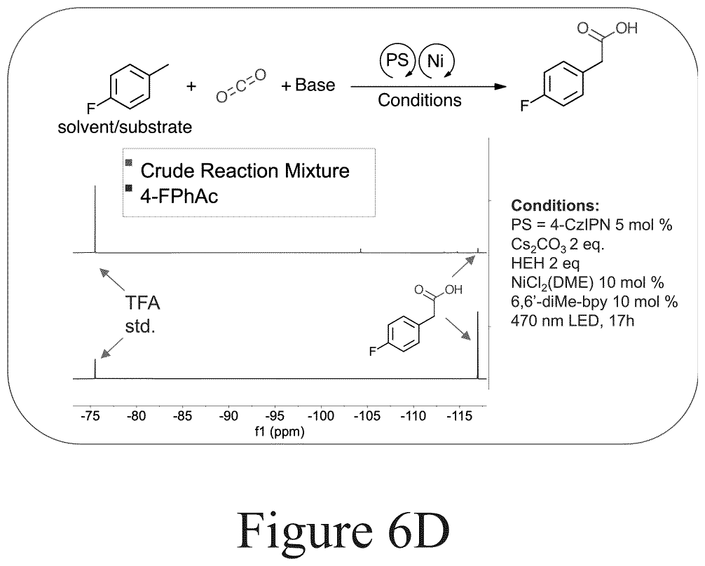

FIG. 7 illustrates UV-vis spectroscopy data obtained for Experimental Compound #5 (see Scheme 16 and Table 1), according to some embodiments of the present disclosure.

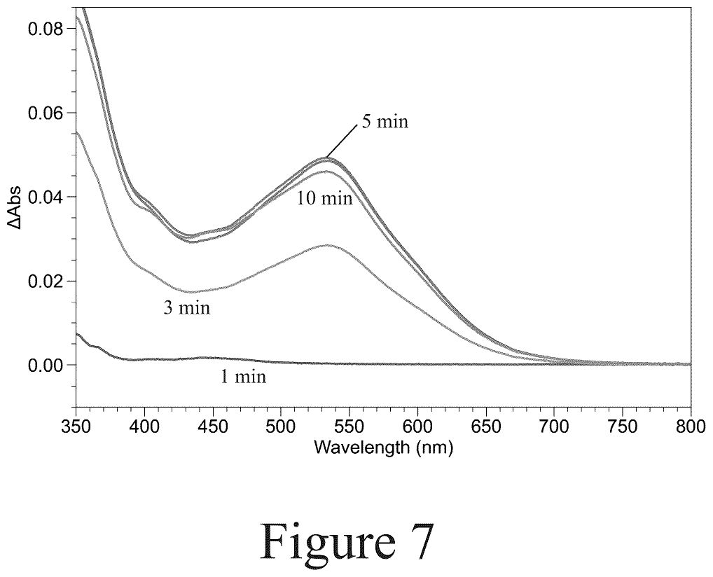

FIG. 8 illustrates ultra-violet visible (UV-vis) spectroscopy data obtained for Experimental Compounds #6a and 6b (see Scheme 16 and Table 1), according to some embodiments of the present disclosure.

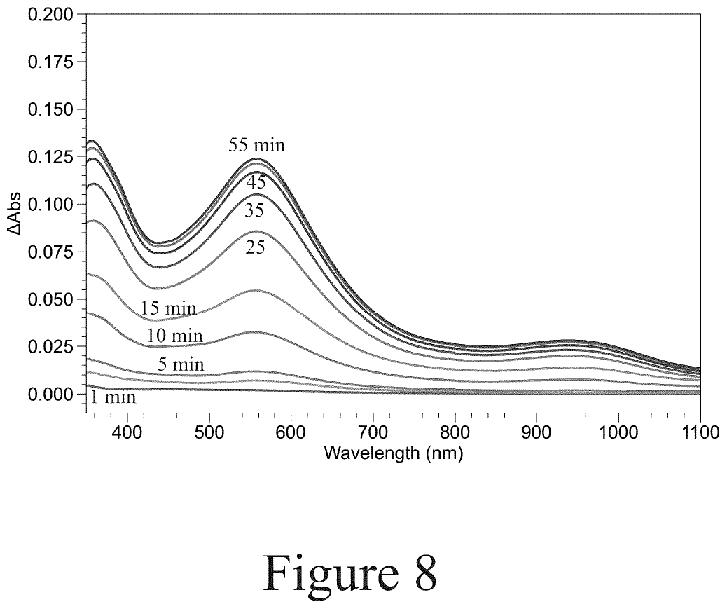

FIG. 9 illustrates ultra-violet visible (UV-vis) spectroscopy data obtained for Experimental Compounds #7a and 7b (see Scheme 16 and Table 1), according to some embodiments of the present disclosure.

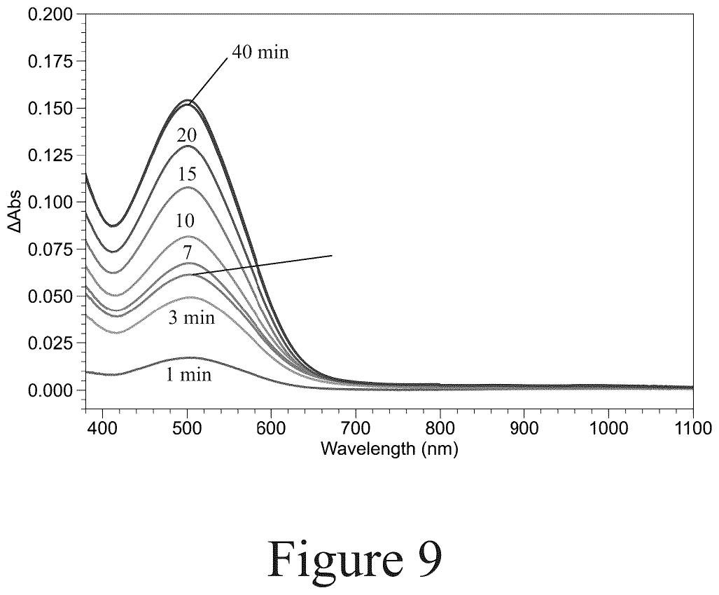

FIG. 10 illustrates ultra-violet visible (UV-vis) spectroscopy data obtained for Experimental Compounds #8a and 8b (see Scheme 16 and Table 1), according to some embodiments of the present disclosure.

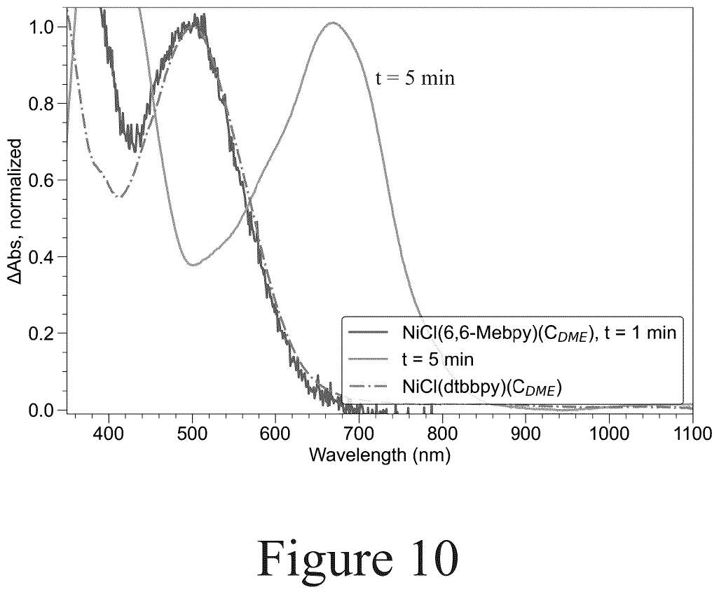

To facilitate understanding, identical reference numerals have been used, where possible, to designate identical elements that are common to the figures. It is contemplated that elements and features of one embodiment may be beneficially incorporated in other embodiments without further recitation.

### REFERENCE NUMBERS

200 . . . method
201 . . . nickel complex
202 . . . ligand
203 . . . nickel pre-catalyst
204 . . . reactant
205 . . . intermediate compound
210 . . . reacting
210A . . . reacting to form pre-catalyst
210B . . . reacting to form intermediate compound
210C . . . reacting to form carboxylic acid
220 . . . carboxylic acid

### DETAILED DESCRIPTION

The present disclosure relates to methods, compositions, compounds, and systems for converting CO₂ and biomass to useful products (e.g., carboxylic acid products) utilizing light and a nickel catalyst. In short, this is achieved by the combination of two reaction steps. First, activation of a C—H bond in the biomass-derived reactant (for example, biomass-derived ethers such as 1,2-dimethoxyethane) is achieved through the photoinduced production of halogen (e.g., chlorine) radicals by the Ni catalyst. The resulting biomass-derived radical species is trapped and bound by the Ni catalyst. Second, light irradiation induces the formation of a Ni(I)—C bond at the Ni catalyst which reacts with CO₂ via insertion resulting in the final products, e.g., carboxylic acids. Note that although biomass-derived reactants are important in view of today's issues regarding global climate change, the compounds and methods described herein are not limited to biomass-derived reactants.

The catalyzed reaction is enabled by combining a light source, a nickel source (i.e., a nickel complex), a ligand, and the biomass-derived reactant. Further, in some embodiments of the present disclosure, the reactant may also serve the purpose of a solvent, enabling the reaction to occur. Further, in some embodiments of the present disclosure, a nickel source may already include the ligand, thus forming the pre-catalyst. Details will be provided below. However, in brief the reaction proceeds as follows. In a first step, a nickel complex and a ligand combine to form a pre-catalyst nickel complex. Irradiation using a light source converts the nickel pre-catalyst into an intermediate nickel compound, containing an organometallic ligand, with the reactant bonded to the nickel atom. Finally, the intermediate compound reacts, resulting in the consumption of CO₂ and the formation of product, e.g., a carboxylic acid.

In some embodiments of the present disclosure, a light source may be a light emitting diode (LED) providing light having a maximum emission wavelength between 300 nm and 800 nm. In some embodiments of the present disclosure, a nickel source may be a Ni(II) dihalide salt, which, when combined with a ligand, results in a nickel pre-catalyst having the generalized stoichiometry NiX₂L, where X is a halogen and L is the ligand, for example NiCl₂(dtbbpy). The dtbbpy (4,4′-di-tert-butyl-2,2′-dipyridyl) ligand can be added separately, in which case an example nickel salt is NiCl₂(DME), where DME is 1,2-dimethoxyethane. In general, a ligand may be a bidentate ligand binding the nickel atom through the use of two nitrogen atoms. A solvent may be an organic solvent that may also act as a substrate/reactant, with an example being DME. An example of a solvent that acts only as a solvent that is inert to the chemistry is benzene, where the reactant is added separately, sometimes at a lower concentration than the solvent.

In some embodiments of the present disclosure, the performance of reactions described above may be improved by the addition of one or more optional components, which may improve the degree of conversion of the pre-catalyst to the intermediate compound. One such optional component is a base, for example 2,6-lutidine. A second optional component is a photosensitizer, for example [Ir(dF(CF₃)ppy₂(dtbbpy)]PF₆ where dF(CF₃)ppy=2-(2,4-difluorophenyl)-5-trifluoromethylpyridine). A third optional component is the introduction of air into the headspace of the reaction vessel. In situations where air is not used in the headspace, the reaction headspace may be a nitrogen atmosphere with CO₂.

With the active nickel catalyst (i.e., intermediate compound) successfully synthesized, it can complete the light-driven conversion reaction of CO₂ and reactant to the target products, e.g., carboxylic acids. The active catalyst may either be prepared in a separate pre-irradiation step and/or it may be formed in a single reaction step by combining all the above components with CO₂ and irradiating with light to form the desired carboxylic acid product. The reaction may result in forming the product as a carboxylate salt with the protonated base. A subsequent step of adding acid may be included, for example 2 M aqueous HCl, to liberate the carboxylic acid product.

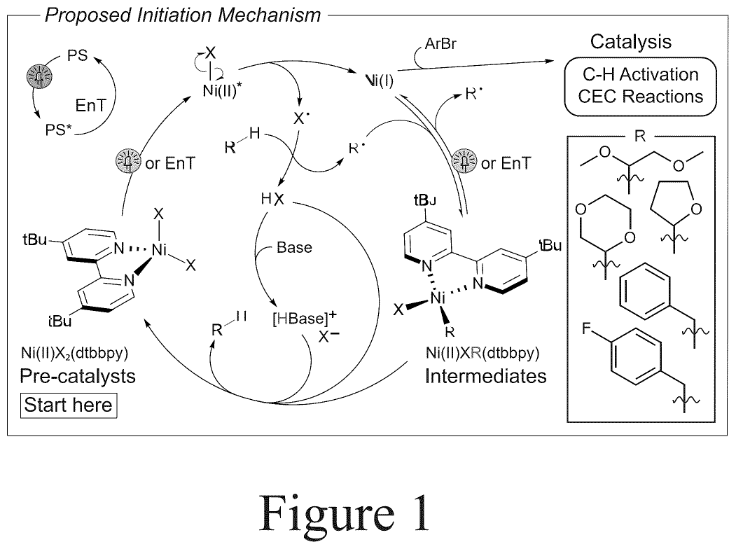

FIG. 1 illustrates a mechanism that explains the reaction steps described above. In brief, an exemplary nickel pre-catalyst, a Ni(II)X₂(dtbbpy), where X=Cl or Br and the ligand L=dtbbpy, in an excited state forms through either photoexcitation and/or energy transfer and undergoes photolysis of the Ni-X bond. The resulting X· abstracts a H-atom from R—H of the reactant to form R· and HX. The HX can be intercepted by a base (if present) to form the base-halide salt, which serves to suppress the protonation that limits buildup of the intermediate compound, Ni(II)XR(dtbbpy). R· recombines with Ni(I)X(dtbbpy) to form Ni(II)XR(dtbbpy) (i.e., the intermediate compound), which is also susceptible to photolysis induced by photoexcitation or energy transfer. As such, Ni(II)XR(dtbbpy) serves as a reservoir of active Ni(I) species that protects the Ni(I), limiting its concentration and thus its propensity to undergo off-cycle dimerization. Lastly, changing L=dtbbpy to L=CF₃bpy or L=66Mebpy increases the ratio of Ni(I)XL to Ni(II)XRL photo-products, thus providing a way to control the buildup of these reactive intermediates to suit a particular catalytic system. The organometallic R ligand in the Ni(II)XRL complex serves as a defining feature which allows that group to be incorporated into a desired product, for example in Ni-catalyzed C—H activation reactions. Alternatively, the R group (the R₂ group referenced below) can be thought of as a mask which can be easily removed to expose an active Ni(I) catalyst, such as Ni(I)XL. As such, the intermediate compound Ni(II) XRL can be used broadly as a pre-catalyst in many Ni-catalyzed reactions that rely on Ni(I) as the active catalyst.

As described herein, an intermediate compound can be isolated and used to catalyze a number of reactions, including the carboxylation of ethers and molecules containing benzyl groups to produce a wide variety of carboxylic acids, including dicarboxylic acids. Dicarboxylic acids are a widely used class of monomer used to make polyesters. Some members of this class, for example terephthalic acid, are commodity chemicals utilized on a very large industrial scale. The dicarboxylic acid is typically co-polymerized with a diol. If the dicarboxylic acid contains an alcohol functional group, the diol is not required, and it can be polymerized to form a polyester. The dicarboxylic acids that can be produced with this method include new compounds, from which novel polyesters could be made, as well as known compounds.

In general, the intermediate compounds described herein have a structure as defined by Structure 1.

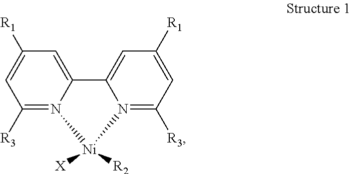

Here, R₁ includes at least one of an alkyl group, a halogen, or a combination thereof. Examples of alkyl groups include methyl, ethyl, propyl, and/or butyl groups, etc. In some embodiments of the present disclosure, R₁ may include an alkyl group may have between 1 and 10 carbon atoms and be either a branched alkyl group and/or a linear alkyl group. In some embodiments of the present disclosure, Ri may include at least one of —C(CH₃)₃, —CF₃, H, or a combination thereof. R₃ includes at least one of hydrogen, an alkyl group, or a combination thereof. Again, suitable examples of alkyl groups for R₃ include methyl, ethyl, propyl, and/or butyl groups, etc. In some embodiments of the present disclosure, R₃ may include an alkyl group may have between 1 and 10 carbon atoms and be either a branched alkyl group and/or a linear alkyl group. X may be a halogen, including at least one of fluorine, chlorine, bromine, iodine, or a combination thereof.

R₂ of Structure 1 may be a functional group that includes at least one of a benzyl group, an ether, or a combination thereof. Example of R₂ with benzyl groups are summarized below in Scheme 1.

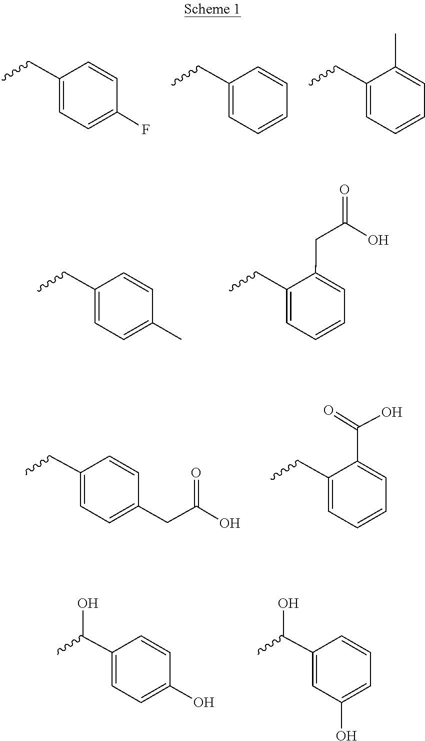

Examples of linear ethers suitable as R₂ groups are summarized in Scheme 2.

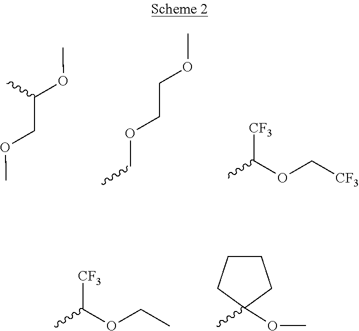

Examples of cyclic ethers suitable as R₂ groups are summarized in Scheme 3.

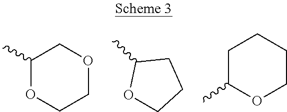

More specific examples of Structure 1 are summarized in Scheme 4, where for Compound A and Compound B, the R₁ groups are tert-butyl groups and trifluoromethyl groups, respectively, and for Compound C, R₃ are methyl groups.

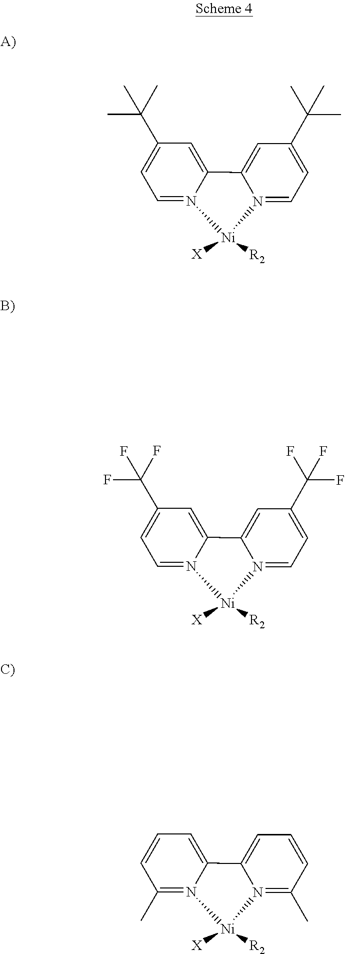

Examples of more specific compounds are illustrated in additional schemes as summarized in Table 1.

TABLE 1
Compounds

| Compound from Scheme 4 | R₂ type | Scheme # |
|---|---|---|
| Compound A | linear ether | 5 |
| Compound A | cyclic ether | 6 |
| Compound A | benzyl groups | 7 |
| Compound B | linear ether | 8 |
| Compound B | cyclic ether | 9 |
| Compound B | benzyl groups | 10 |
| Compound C | linear ether | 11 |
| Compound C | cyclic ether | 12 |
| Compound C | benzyl groups | 13 |

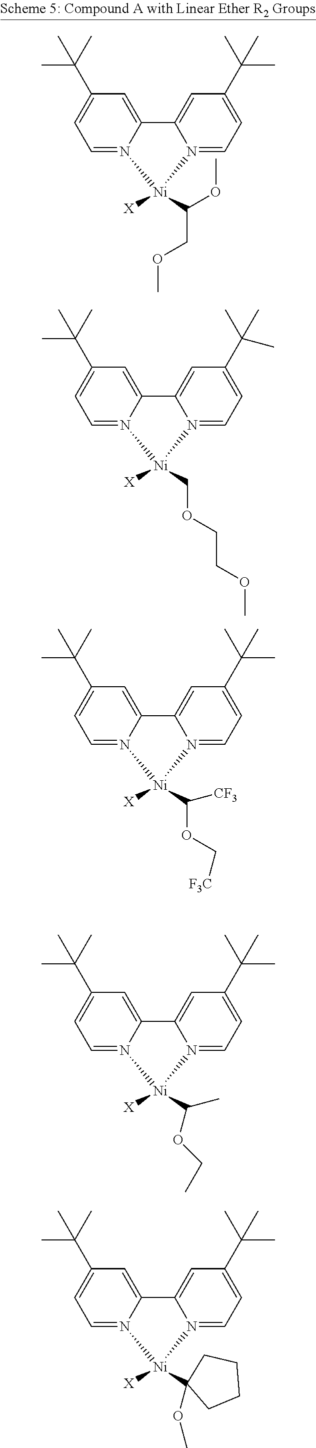

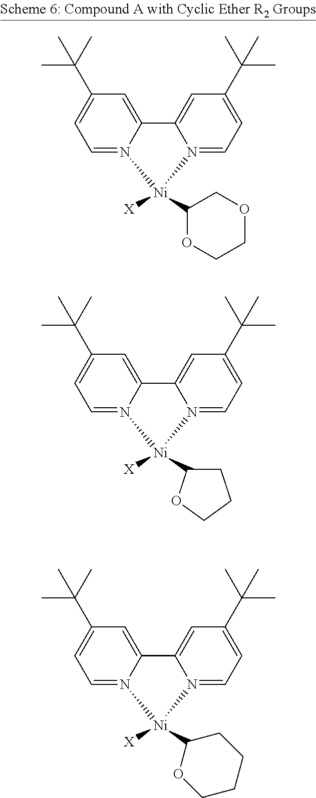

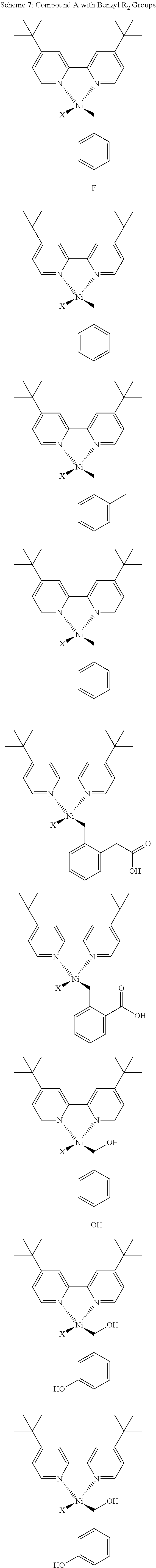

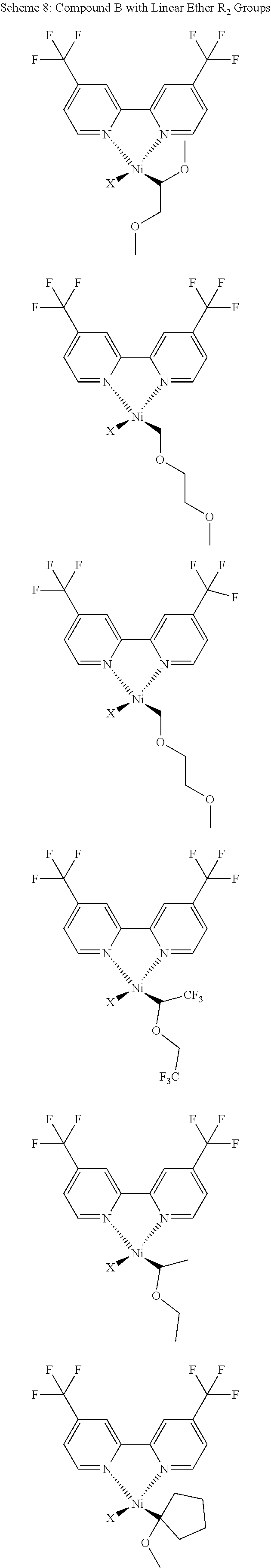

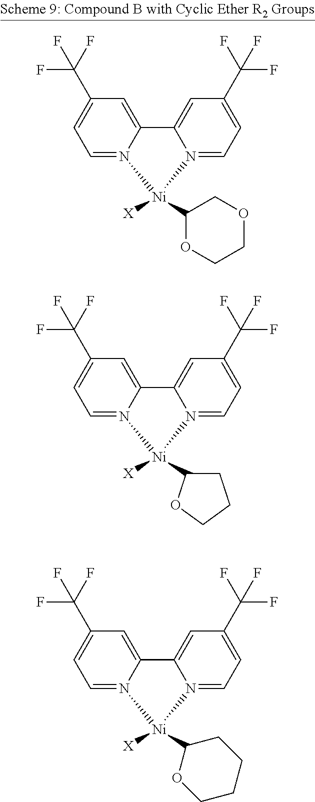

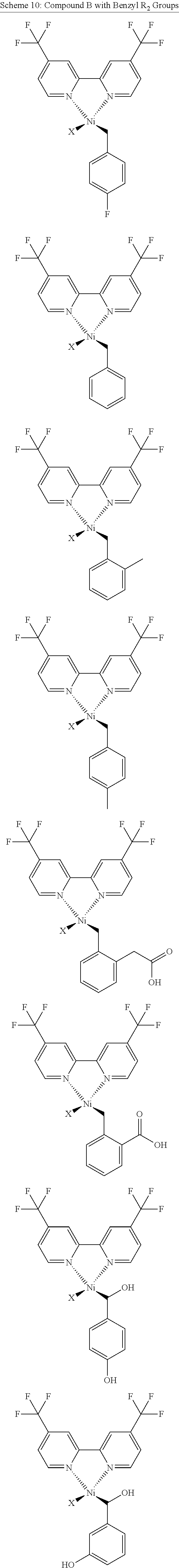

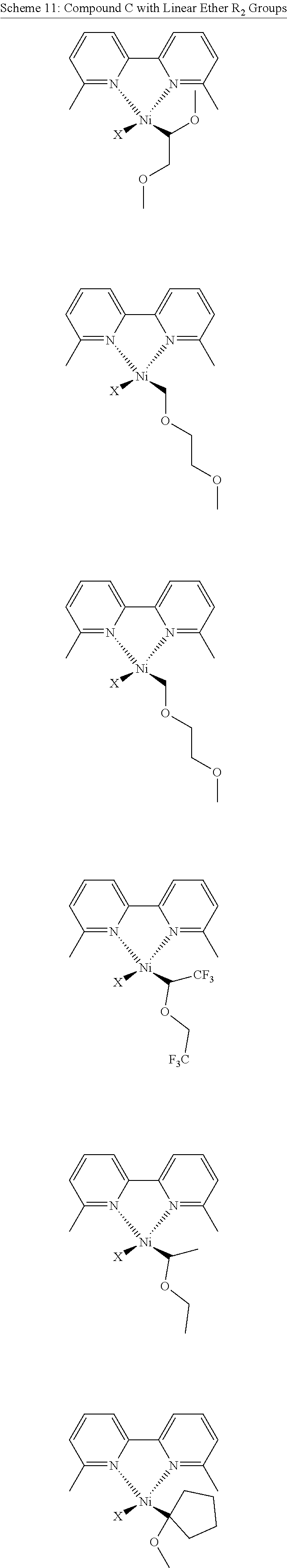

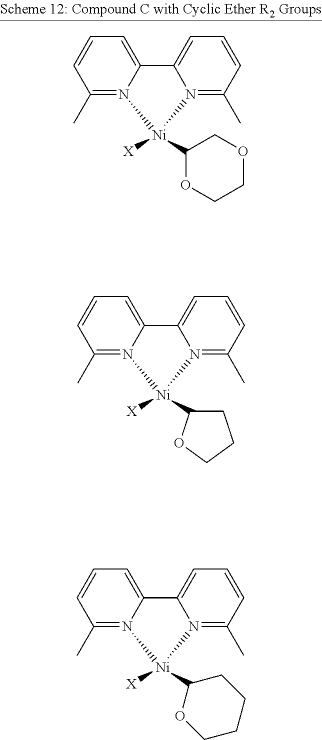

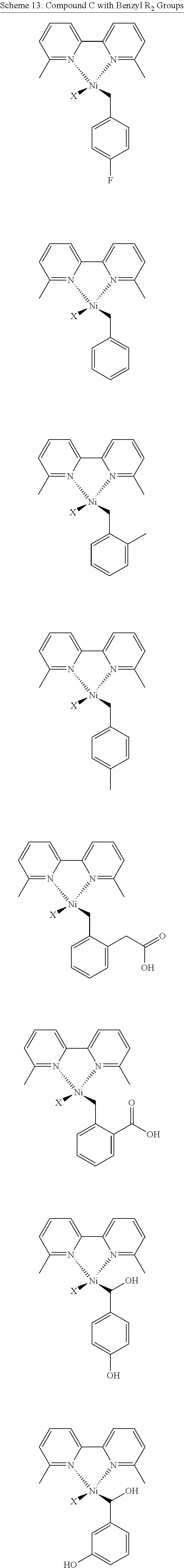

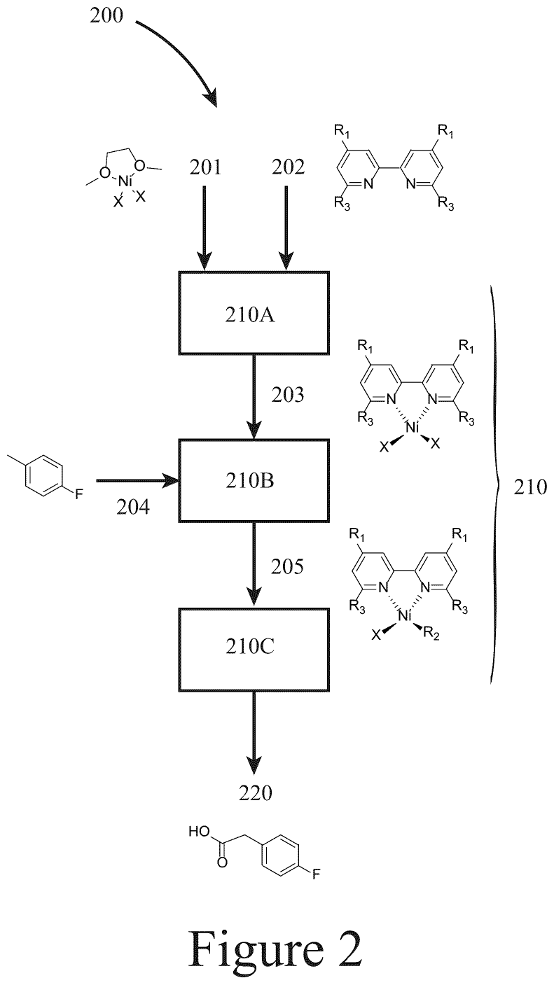

FIG. 2 illustrates a method 200 for making both the compounds discussed above and illustrated in Structure 1 and Schemes 1-13, and how such compounds may be utilized to manufacture useful products, e.g., carboxylic acids. In general, the method 200 illustrated includes reacting CO₂ (not shown) and a nickel pre-catalyst 203 with a reactant 204, in the presence of light (not shown) to produce a product, a carboxylic acid 220. As described above and illustrated in FIG. 1, a reactant 204 may combine with a nickel pre-catalyst 203 to form an intermediate compound 205 as discussed above and illustrated in Structure 1 and Schemes 1-13. Thus, in some embodiments of the present disclosure, a reactant 204 may include at least one of a benzyl group, an ether, or a combination thereof, resulting in the synthesis of a carboxylic acid having at least one of a benzyl group, an ether, or a combination thereof.

Referring again to FIG. 2, the method 200 illustrated includes a reacting 200 step, that as described above and illustrated in FIG. 1, may include three steps. These steps may be performed in series and essentially simultaneously as described above and illustrated in FIG. 1. Thus, reacting 210 may start with a first step, 210A, where a nickel complex 201 reacts with a ligand 202 to produce a nickel pre-catalyst 203. The general structure of a ligand 202 is shown as Structure 2, an example of a nickel complex 201 is shown in Structure 3, and an example of a nickel pre-catalyst 203 is shown in Structure 4.

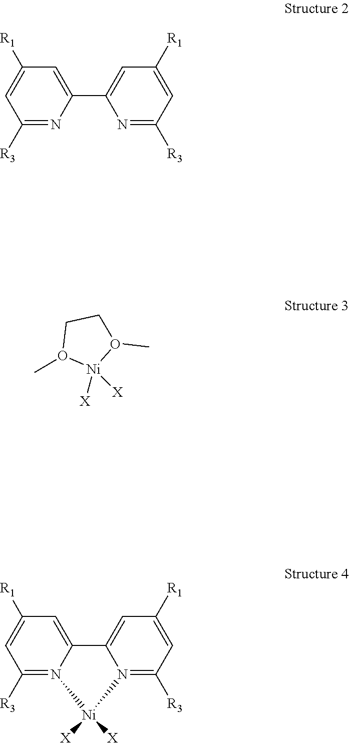

Each of R₁, R₂, R₃, and X illustrated in Structures 2-4 are identical to those described above for Structure 1.

Referring again to FIG. 2, the reacting 210 may continue with a second reacting step 210B, where the nickel pre-catalyst 203 produced in 210A reacts with the reactant 204 to form an intermediate compound 205, as discussed above and illustrated in Structure 1 and Schemes 1-13. Then, through the interaction of light and the presence of CO2, the intermediate compound reacts in a third step 210C resulting in dissociation of carboxylic acids 220. Addition of dilute acid, for example 2 M HCl, can be included to aid in dissociation of the carboxylic acid. Referring again to FIG. 2, a nickel pre-catalyst 203 and/or an intermediate compound 205 may be irradiated with light having a wavelength between 300 nm and 800 nm or between 350 nm and 500 nm.

Examples of reactants 204 are summarized in Scheme 14 and examples of carboxylic acids 220 are summarized in Scheme 15.

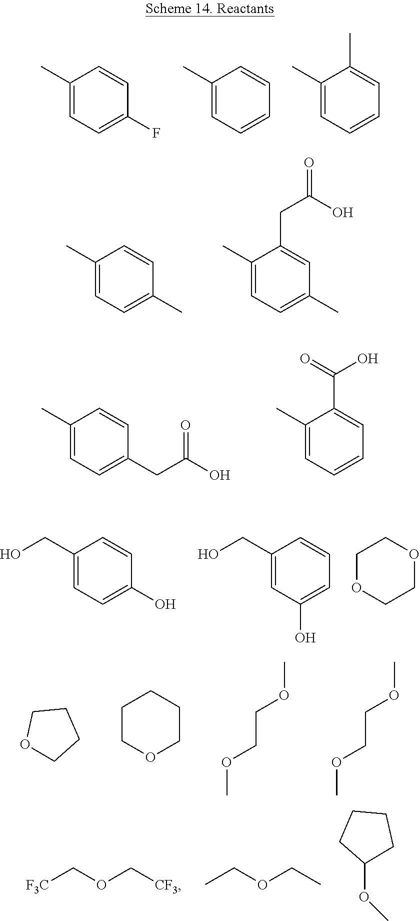

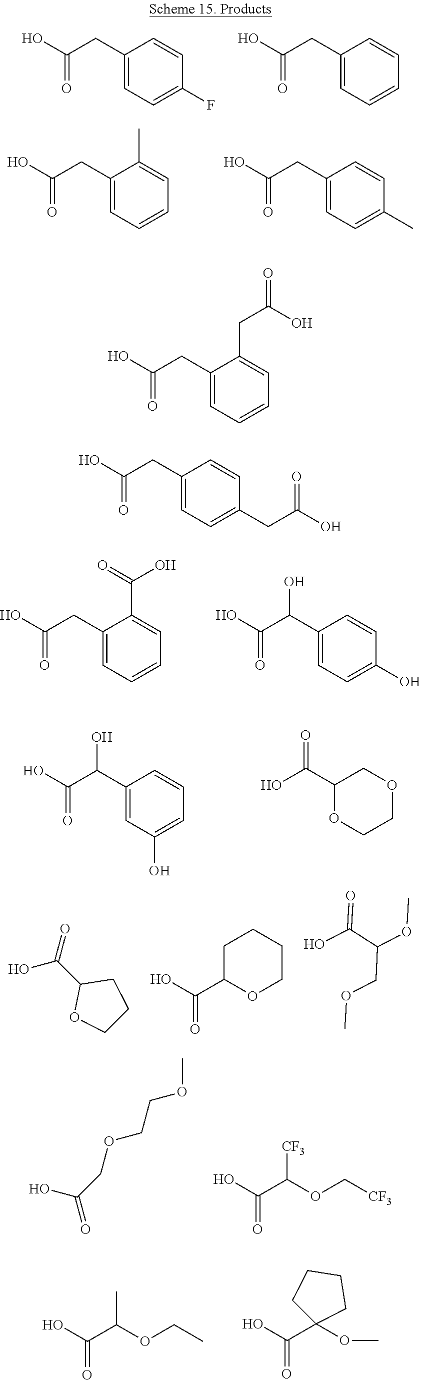

Further, the reacting 210 illustrated in FIG. 2 may include one or more optional components to improve the performance of the reacting 210. These include photosensitizers, bases. and/or sacrificial reductants (not shown). For example, in some embodiments of the present disclosure, reacting 210 may include a photosensitizer (not shown) such as a cyanoarene. Examples of cyanoarene include of 1,2,3,5-tetrakis(carbazol-9-yl)-4,6-dicyanobenzene, 2,4,6-tri(9H-carbazol-9-yl)-5-chloroisophthalonitrile, 2,4,6-tris(diphenylamino)-3,5-difluorobenzonitrile, and 2,4,6-tris (diphenylamino)-5-fluoroisophthalonitrile.

In some embodiments of the present disclosure, reacting 210 may include a base (not shown) such as a carbonate, a phosphate, a tertiary amine, or a combination thereof. Examples of carbonates include Cs₂CO₃, K₂CO₃, and Na₂CO₃. An exemplary phosphate is K₃PO₄. Examples of tertiary amine include 2,6-dimethylpyridine, triethylamine, and 1,4-diazabicyclo[2.2.2]octane. In some embodiments of the present disclosure, reacting 210 may include a sacrificial reductant, with examples including diethyl 1,4-dihydro-2,6-dimethyl-3,5-pyridinedicarboxylate (HEH), triethylamine, and triethanolamine.

Referring again to FIG. 2, in some embodiments of the present disclosure, reacting 210 may be performed using between 380 Torr and 7,600 Torr CO₂ or between 760 Torr and 1,520 Torr CO₂. In some embodiments of the present disclosure, reacting 210 may be performed at a temperature between 0° C. and 200° C. or between 0° C. and 100° C. In some embodiments of the present disclosure, reacting 210 may be performed at a concentration of nickel relative to the reactant between 0.01 mol % and 30 mol % or between 0.05 mol % and 15 mol %. In some embodiments of the present disclosure, reacting 210 may be performed at a concentration of ligand relative to the reactant between 0.001 mol % and 60 mol % or between 0.01 mol % and 30 mol %. In some embodiments of the present disclosure, reacting 210 may be performed at a concentration of photosensitizer relative to the reactant between 0.001 mol % and 60 mol % or between 0.01 mol % and 30 mol %. In some embodiments of the present disclosure, reacting 210 may be performed at a concentration of sacrificial reductant between 0.01 eq (relative to the reactant) and 10 eq or between 0.1 eq and 3 eq. In some embodiments of the present disclosure, reacting 210 may be performed at a concentration of base between 0.01 eq and 10 eq or between 0.1 eq and 3 eq.

### Experimental

### Proof of Synthesis of Intermediate Compounds

A number of intermediate compounds 205 and resultant carboxylic acids were successfully synthesized in the laboratory. The intermediate compounds synthesized, each with a unique identifying number are summarized in Scheme 16. The locating of the experimental results supporting their synthesis are summarized in Table 2. These are primarily UV-vis, NMR, and XRD data.

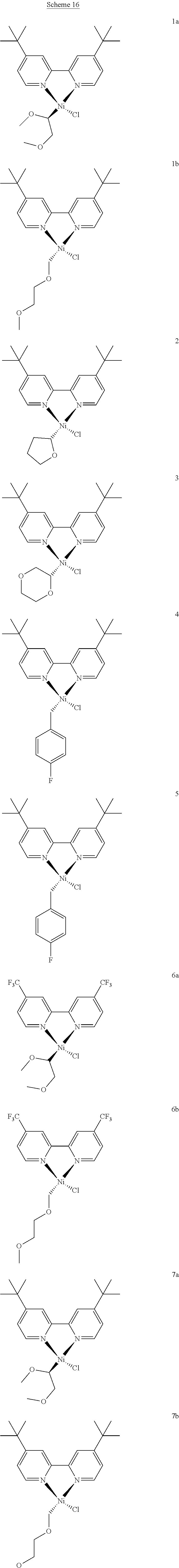

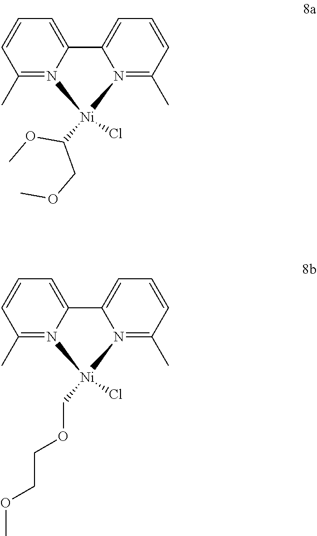

TABLE 2
Location of Experimental Results

| Compound # | FIG. # | Data Type |
|---|---|---|
| 1a, 1b | 3A and 3B | UV-vis; NMR |
| 2 | 4 | UV-vis |
| 3 | 5 | UV-vis |
| 4 | 6A, 6B, 6C | NMR; UV-vis; XRD |
| 5 | 7 | UV-vis |
| 6a, 6b | 8 | UV-vis |
| 7a, 7b | 9 | UV-vis |
| 8a, 8b | 10 | UV-vis |

Photochemical Synthesis of Compound 1 (mixture of 1a/1b isomers). In a N₂-filled glovebox, a a 3.00 mL mixture was prepared in a 1 cm quartz cuvette. To solid nickel pre-catalyst NiCl₂(dtbbpy) (6.6 mg) was added 10 mL of the reactant 1,2-dimethoxyethane in a volumetric flask. The resulting solution was stirred for 24 hours and noted to be saturated. After syringe filtration, 2.7 mL of this solution was mixed with 0.3 mL of a stock solution containing the photosensitizer “Ir PS”, [Ir[dF(CF₃)ppy]₂(dtbbpy)]PF₆, to make a final concentration of the Ir PS of 0.134 mM in 1,2-dimethoxyethane. The mixture was irradiated with a 405 nm LED (216 mW cm⁻²). After each irradiation period, the LED was turned off, the cuvette was transferred to the Cary 7000 spectrometer, and UV-vis was acquired. UV-vis: visible λₘₐₓ=500 nm.

Synthesis of Compound 1 (mixture of 1a/1b isomers) by pulse radiolysis and characterization by NMR and X-ray Absorption Spectroscopy. A mixture was prepared by mixing equal molar ratios of Ni(cod)₂, where cod=1,4-cyclooctadiene, and the pre-catalyst NiCl₂(dtbbpy) to prepare a solution 2.5 mM in total Ni, dissolved in 1,2-dimethoxyethane. This solution was subjected to pulse radiolysis which resulted in formation of Compound 1a/1b.

Photochemical Synthesis of Compound 2. In a N₂-filled glovebox, a 3.00 mL mixture was prepared in a 1 cm quartz cuvette. To the solid pre-catalyst NiCl₂(dtbbpy) (0.8 mg) was added 1.5 mL tetrahydrofuran. To 0.5 mg of solid photosensitizer “Ir PS”, [Ir[dF(CF₃)ppy]₂(dtbbpy)]PF₆, was added 1.5 mL of reactant tetrahydrofuran. The solutions were stirred for 24 hours at room temperature, noted to be fully dissolved, and mixed and transferred to the cuvette (final concentration 2.00 mM in Ni and 0.150 mM in Ir PS). The mixture was irradiated with a 405 nm LED (189 mW cm⁻²). After each irradiation period, the LED was turned off, the cuvette was transferred to the Cary 7000 spectrometer, and UV-vis was acquired. UV-vis: visible λₘₐₓ=515 nm.

Photochemical Synthesis of Compound 3. In a N₂-filled glovebox, a 3.00 mL mixture was prepared in a 1 cm quartz cuvette. First, to solid pre-catalyst NiCl₂(dtbbpy) (2.6 mg was added 4 mL of the reactant 1,4-dioxane, which was stirred for 24 h to make a saturated solution and then syringe filtered. 2.7 mL of the filtered solution was mixed with 0.3 mL of a stock solution containing the photosensitizer “Ir PS”, [Ir[dF(CF₃)ppy]₂(dtbbpy)]PF₆, to dilute it 0.134 mM in 1,4-dioxane. The mixture was irradiated with a 405 nm LED (216 mW cm⁻²). After each irradiation period, the LED was turned off, the cuvette was transferred to the Cary 7000 spectrometer, and UV-vis was acquired. UV-vis: visible λₘₐₓ=477 nm

Synthesis of Compound 4 from Ni(0). In a N₂-filled glovebox, Ni(cod)₂, where cod=1,4-cyclooctadiene (27.5 mg, 0.1 mmol, 1.0 eq.) was weighed into a 4 mL vial and stirred in 2 mL benzene for 15 minutes. The ligand dtbbpy (26.8 mg, 0.1 mmol, 1.0 eq.) was weighed into a second 4 mL vial and dissolved fully in 2 mL benzene. The dtbbpy solution was transferred to the vial containing Ni(cod)₂, and a color change from yellow to dark purple was observed as the solution was stirred for another 15 minutes until fully dissolved. This dark purple solution was then transferred to a 4 mL vial containing 4-fluorobenzyl chloride (0.048 mL, 0.4 mmol, 4.0 eq.). An immediate color change to dark red was noted. After 15 minutes, the solution was added slowly to 5 mL anhydrous n-pentane in a 20 mL vial, which resulted in precipitation of a dark red solid. The solid was collected in a 25 mm PTFE syringe filter (Avantor) and rinsed 2x with 2 mL n-pentane, which resulted in more precipitation. The filtrate was filtered again and the filter was rinsed 2x with 2 mL n-pentane. The two filters were dried under vacuum. One filter was noted to contain crystalline material and was set aside. For characterization, the dried solid trapped in the filters was re-dissolved in the solvent of choice. Appearance: dark red powder; Yield: 32.1 mg; 68%; SCXRD: a dark red translucent rhomboid plate was subjected to crystallographic analysis, and the structure found agrees with that shown above.

Photochemical Synthesis of Compound 4. In a N₂-filled glovebox, a mixture was prepared in a 1 cm quartz cuvette. To solid pre-catalyst NiCl₂(dtbbpy) (0.5 mg) was added 2.7 mL of reactant 4-fluorotoluene and the resulting solution was stirred for 24 hours. A second solution containing the photosensitizer “Ir PS”, [Ir[dF(CF₃)ppy]₂(dtbbpy)]PF₆ (1.5 mg), was prepared by adding 1.0 mL of 4-flurotoluene and stirring for 24 hours. Both saturated solutions of Ni and the Ir PS were syringe filtered and 2.7 mL of the filtered Ni solution was mixed with 0.3 mL of the filtered Ir PS solution. The mixture was irradiated with a 405 nm LED (189 mW cm⁻²). After each irradiation period, the LED was turned off, the cuvette was transferred to the Cary 7000 spectrometer, and UV-vis was acquired. UV-vis: visible λₘₐₓ=514 nm.

Photochemical Synthesis of Compound 5. In a N₂-filled glovebox, a a 3.00 mL mixture was prepared in a 1 cm quartz cuvette. To solid pre-catalyst NiCl₂(dtbbpy) (6.6 mg) was added 10 mL of reactant toluene in a volumetric flask. The resulting solution was stirred for 24 hours and noted to be saturated. After syringe filtration, 2.7 mL of this solution was mixed with 0.3 mL of a stock solution (1.5 mg in 1 mL toluene) containing the photosensitizer “Ir PS”, [Ir[dF(CF₃)ppy]₂(dtbbpy)]PF₆, to make a final concentration of the Ir PS of 0.134 mM in 1,2-dimethoxyethane. The mixture was irradiated with a 405 nm LED (216 mW cm⁻²). After each irradiation period, the LED was turned off, the cuvette was transferred to the Cary 7000 spectrometer, and UV-vis was acquired. UV-vis: visible λₘₐₓ=530 nm

Synthesis of Compound 5 from Compound 4. Compound 4 was converted to Compound 5 spontaneously at room temperature by dissolution in reactant toluene (3.1 mg in 1 mL).

Photochemical Synthesis of Compound 6 (mixture of isomers 6a/6b). In a N₂-filled glovebox, a a 3.00 mL mixture was prepared in a 1 cm quartz cuvette. To solid nickel complex NiCl₂(DME) (0.8 mg) was added 2.0 mL of reactant 1,2-dimethoxyethane (DME). The ligand 4,4′-ditrifluoromethyl-2,2′-bipyridine was weighed and dissolved in 0.7 mL DME. The solutions of Ni and ligand were combined and the resulting solution containing the pre-catalyst was stirred for 24 hours and noted to be dissolved. 2.7 mL of this solution was mixed with 0.3 mL of a stock solution (1.5 mg in 1 mL DME) containing the photosensitizer “Ir PS”, [Ir[dF(CF₃)ppy]₂(dtbbpy)]PF₆, to make a final concentration of the Ir PS of 0.134 mM in 1,2-dimethoxyethane. 2,6-lutidine (10 eq. relative to Ni, 10 mM, 3.5 μL) was added. The mixture was irradiated with a 405 nm LED (189 mW cm⁻²). After each irradiation period, the LED was turned off, the cuvette was transferred to the Cary 7000 spectrometer, and UV-vis was acquired. UV-vis: visible λₘₐₓ=558 nm.

Photochemical Synthesis of Compound 7 (mixture of isomers 7a/7b). In a N₂-filled glovebox, a a 3.00 mL mixture was prepared in a 1 cm quartz cuvette. To solid nickel complex NiBr₂(DME) (4.4 mg) was added 1.7 mL of reactant 1,2-dimethoxyethane (DME). The ligand 4,4′-di-tert-butyl-2,2′-bipyridine (dtbbpy) was weighed (4.1 mg) and dissolved in 1.0 mL DME. The solutions of nickel complex and ligand were combined and the resulting pre-catalyst solution was stirred for 24 hours and noted to be dissolved. 2.7 mL of this solution was mixed with 0.3 mL of a stock solution containing the photosensitizer “Ir PS”, [Ir[dF(CF₃)ppy]₂(dtbbpy)]PF₆, to make a final concentration of the Ir PS of 0.150 mM in 1,2-dimethoxyethane. The mixture was irradiated with a 405 nm LED (216 mW cm⁻²). After each irradiation period, the LED was turned off, the cuvette was transferred to the Cary 7000 spectrometer, and UV-vis was acquired. UV-vis: visible λₘₐₓ=500 nm.

Photochemical Synthesis of Compound 8 (mixture of isomers 8a/8b). In a N₂-filled glovebox, a a 3.00 mL mixture was prepared in a 1 cm quartz cuvette. To solid nickel complex NiCl₂(DME) (0.8 mg) was added 2.0 mL of reactant 1,2-dimethoxyethane (DME). The ligand 6,6′-dimethyl-2,2′-bipyridine was weighed and dissolved in 0.7 mL DME. The solutions of nickel complex and ligand were combined and the resulting pre-catalyst solution was stirred for 24 hours and noted to be dissolved. 2.7 mL of this solution was mixed with 0.3 mL of a stock solution (1.5 mg in 1 mL DME) containing the photosensitizer “Ir PS”, [Ir[dF(CF₃)ppy]₂(dtbbpy)]PF₆, to make a final concentration of the Ir PS of 0.134 mM in 1,2-dimethoxyethane. The base 2,6-lutidine (10 eq. relative to Ni, 10 mM, 3.5 μL) was added. The mixture was irradiated with a 405 nm LED (189 mW cm⁻²). After each irradiation period, the LED was turned off, the cuvette was transferred to the Cary 7000 spectrometer, and UV-vis was acquired.

### Supporting Evidence for Carboxylation of 4-Fluorotoluene

Reaction Procedure. In a N₂-filled glovebox, 1.5 mg (0.05 eq., 0.008 mmol) of the ligand 6,6′-dimethyl-2,2′-bipyridine (6,6′-Mebpy) was weighed into a 4 mL vial and dissolved in 200 μL of reactant 4-fluorotoluene. 3.16 mg (0.05 eq., 0.004 mmol) of the photosensitizer 1,2,3,5-Tetrakis(carbazol-9-yl)-4,6-dicyanobenzene (4CzIPN) was added to a second vial and dissolved in 400 μL 4-fluorotoluene. The nickel complex NiBr2 (DME) was weighed into a third vial (2.5 mg, 0.05 eq., 0.008 mmol) and dissolved in 400 μL 4-fluorotoluene. The 6,6′-Mebpy ligand solution was combined with the nickel complex NiBr2 (DME) solution and allowed to stir for 30 minutes to form the pre-catalyst at which point the photosensitizer 4CzIPN solution was added. The resulting solution was transferred to a vial containing 40.5 mg (2.0 eq., 0.16 mmol) of sacrificial reductant diethyl 1,4-dihydro-2,6-dimethyl-3,5-pyridinedicarboxylate (HEH) and stirred for 30 minutes. The resulting solution was syringe filtered into a 1.5 mL vial charged with a stir bar and containing the base Cs2CO3 (52.1 mg, 2.0 eq., 0.16 mmol). The completed solution was removed from the glovebox and irradiated with a 470 nm LED (155 mW) while stirring for 17 hours. The solvent was removed under vacuum and the crude solid residue was dissolved in CDCl₃ for NMR analysis.

### Experimental Procedures

UV-visible spectroscopy (UV-vis). A Varian Cary-7000 UV-Vis spectrophotometer was used to acquire absorbance spectra. Spectra were acquired scanning over the indicated range with wavelength resolution of 1 nm at a rate of 600 nm/min.

Nuclear Magnetic Resonance (NMR) Spectroscopy. All ¹H NMR data were collected on a 600 MHz (14.7 Tesla) Bruker Avance NEO NMR spectrometer equipped with a 5 mm iProbe tuned to ¹H. The same instrument and probe was utilized for ¹⁹F and ¹³C experiments. Experiments were performed with samples dissolved in a mixed 1:10 v/v DME or 4FTol:benzene-de solvent system prepared in a N₂-filled glovebox in a 5 mm NMR tube fitted with a J-Young type teflon screw cap closure (Wilmad). Samples were irradiated in the custom photoreactor setup in the NMR tube, removed, and immediately measured. A manual inject/eject routine was utilize to minimize downtime between irradiation and measurement. Due to the high concentration of DME in the samples, a collection protocol with the following parameters was developed to avoid detector saturation. All samples were collected with a 10 degree tip angle, a receiver gain of 90.5, a relaxation delay of 1 s, an acquisition time of 2.75 s, and 64 scans. Shimming was completed with the topshim command with ordmax=5, tunebz, and tuneaz keywords. MestReNova software was utilized for all data analysis. For spectra of isolated organonickel complexes, pure benzene-d6 was utilized. For ¹⁹F NMR, a flame-sealed capillary tube containing trifluoroacetic acid in D₂O was inserted into each sample and used to manually reference ¹⁹F NMR shifts with the literature reported² chemical shift value for TFA in benzene-d₆. For each spectrum, the “Automatic Baseline Correction” and “Automatic Phase Correction” functions were applied. Chemical shifts for ¹H and ¹³C were referenced relative to the benzene impurity in benzene-d₆.

Pulse Radiolysis Studies. A detailed description of the Laser-Electron Accelerator Facility (LEAF) at Brookhaven National Laboratory has been presented previously.⁴ All the samples were prepared in degassed anhydrous DME as the solvent to a total volume of 250 μL inside an Ar-filled glovebox. These were further transferred to a 0.5 cm path length square quartz cuvette with a J. Young style air-tight sealing mechanism in the glove box. Samples were subjected to irradiation directly following their preparation, taking precautions to minimize ambient light exposure and time spent outside of the glovebox during the process. The samples were exposed to <50 ps pulses of 9 MeV electrons. The laser pulses produce a typical concentration of radical anions on the order of 1 μM. The transient absorption was measured using a pulsed xenon lamp using a silicon (EG&G FND-100Q, <1000 nm) detector depending on the wavelength. Wavelengths were selected using interference filters placed before the detector. The detector output was digitized with a LeCroy Waverunner 640Zi.

X-ray Absorption Spectroscopy (XAS). Nickel K-edge XAS spectra were acquired at beamlines 4-1 and 9-3 of the Stanford Synchrotron Radiation Lightsource (SSRL). Mixtures of LED-irradiated Ni/Ir(Air) and Ni/Ir/lutidine(N₂) samples (BL 4-1) prepared as described in the X-Ray Absorption Section below were measured at room temperature under continuous flow (without recycling) using a PEEK liquid flow cell with air-tight Kapton film cover. All components of the flow cell were dried under glove box antechamber vacuum prior to use, and the cell and tubing was flushed with argon at the beamline prior to injecting the sample, which was filtered inline through a 0.2 micron PTFE syringe filter. Between each sample, the Kapton window was replaced, then the cell was rinsed with dry DME between samples and sparged with argon. An analogous setup was used to measure commercially-sourced (Sigma-Aldrich) Ni(dtbbpy)Cl₂ as a saturated solution in DME at BL 9-3. A solid sample of NiCl(TMEDA)(o-tolyl) (TMEDA=N,N,N′,N′-Tetramethylethane-1,2-diamine, o-tolyl=ortho-tolyl) (BL 4-1) purchased from STREM Chemicals was prepared in a nitrogen-filled glove box by diluting the solid to 10% by mass of metal complex in boron nitride (Sigma Aldrich), then packing the solid mixture into a metal frame covered on both sides with Kapton tape. The sample was then sealed in plastic prior to removal from glove box.

Each sample window was positioned at a 45 degree angle relative to the incident beam, whose X-ray energy was modulated using a Si(220) monochromator. Spectra of irradiated Ni/Ir were recorded using a Canberra 30-element Ge solid-state detector, while spectra of NiCl(TMEDA)(o-tolyl) and Ni(dtbbpy)Cl₂ were recorded using a Lytle detector. All detectors were located after a Soller slit and Co filter to exclude elastic scatter, and all spectra are reported in total fluorescence yield. Nickel foil XAS spectra were collected in transmission mode, and the energies were calibrated to the Ni foil first derivative peak energy set to 8333 eV.

The first scan is reported for the Ni/Ir (air) and Ni/Ir/lut (N₂) mixtures since decomposition was observed in subsequent scans (See X-ray Absorption Spectroscopy Section below for further details). An average of two scans is reported for NiCl(TMEDA)(o-tolyl) and Ni(dtbbpy)Cl₂. Data were processed using Athena, where background subtraction, flattening, and an edge jump normalization to 1 for all spectra was applied.

### Examples

Example 1. A compound comprising:

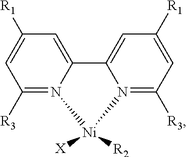

wherein: R₁ comprises at least one of a methyl group, a halogen, or a combination thereof, R₂ comprises at least one of a benzyl group, an ether, or a combination thereof, R₃ comprises at least one of hydrogen, a methyl group, or a combination thereof, and X comprises a halogen. R₂ comprises a covalent bond between Ni and C, where the C comprising the bond is not part of an aromatic ring. Example 1a. The compound of Example 1, further comprising an alkyl linking group positioned between the nickel atom and R₂.

Example 1b. The compound of Example 1a, wherein the alkyl group is a —CH₂-group.

Example 2. The compound of Example 1, wherein R₁ comprises at least one of —C(CH₃)₃, —CF₃, H, or a combination thereof.

3. The compound of either Example 1 or Example 2, wherein X comprises at least one iodine, chlorine, bromine, or a combination thereof.

Example 4. The compound of any one of Examples 1-3, wherein the benzyl group is selected from the group consisting of

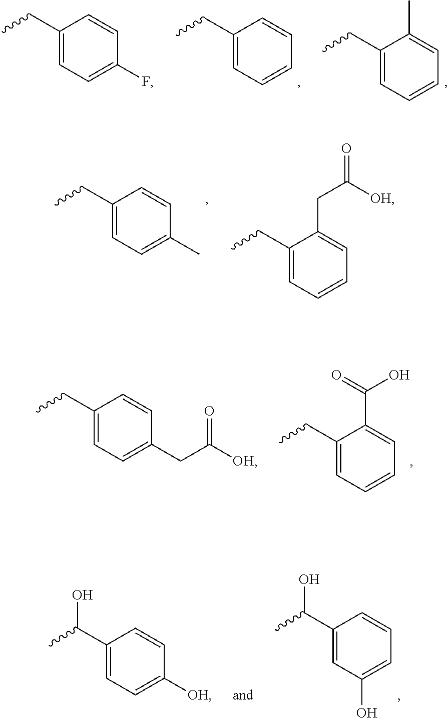

and [symbol] is a covalent bond between R₂ and Ni.

Example 5. The compound of any one of Examples 1-4, wherein the ether is a linear molecule.

Example 6. The compound of any one of Examples 1-5, wherein the linear molecule is selected from the group consisting of

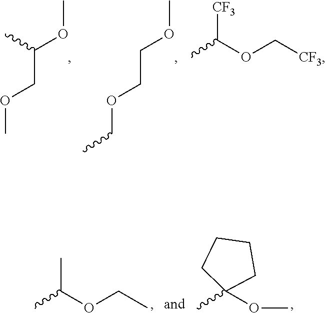

and [symbol] is a covalent bond between R₂ and Ni.

Example 7. The compound of any one of Examples 1-6, wherein the ether is a cyclic molecule.

Example 8. The compound of any one of Examples 1-7, wherein the cyclic molecule is selected from the group consisting of

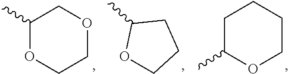

and [symbol] is a covalent bond between R₂ and Ni.

Example 9. The compound of any one of Examples 1-8 comprising

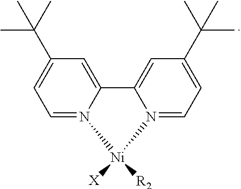

Example 10. The compound of any one of Examples 1-9, wherein R₂ comprises a linear ether.

Example 11. The compound of any one of Examples 1-10, comprising at least one of

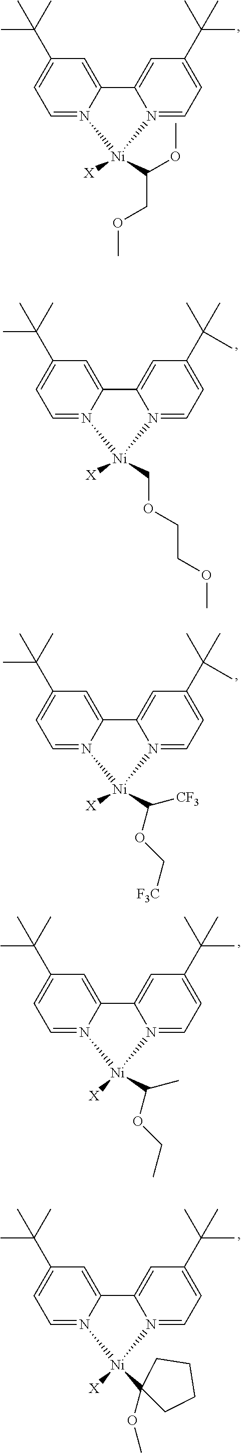

or a combination thereof.

Example 12. The compound of any one of Examples 1-11, wherein X is chlorine.

Example 13. The compound of any one of Examples 1-12, wherein R₂ comprises a cyclic ether.

Example 14. The compound of any one of Examples 1-13, comprising at least one of

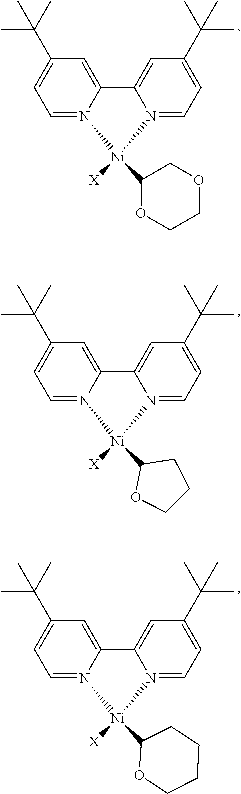

or a combination thereof.

Example 15. The compound of any one of Examples 1-14, wherein X is chlorine.

Example 16. The compound of any one of Examples 1-15, wherein R₂ comprises a benzyl group.

Example 17. The compound of any one of Examples 1-16, comprising at least one of

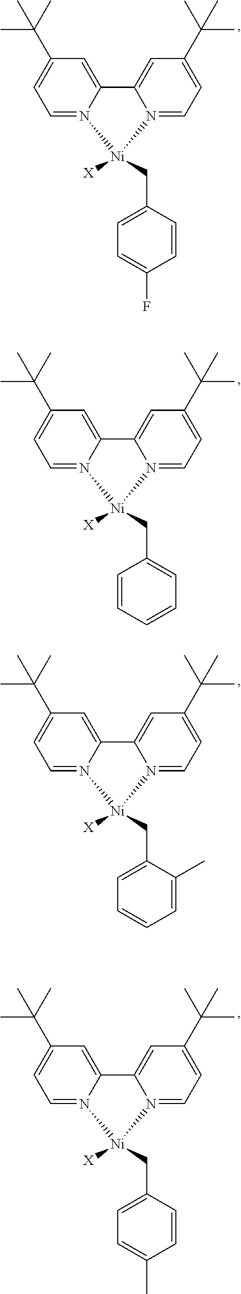

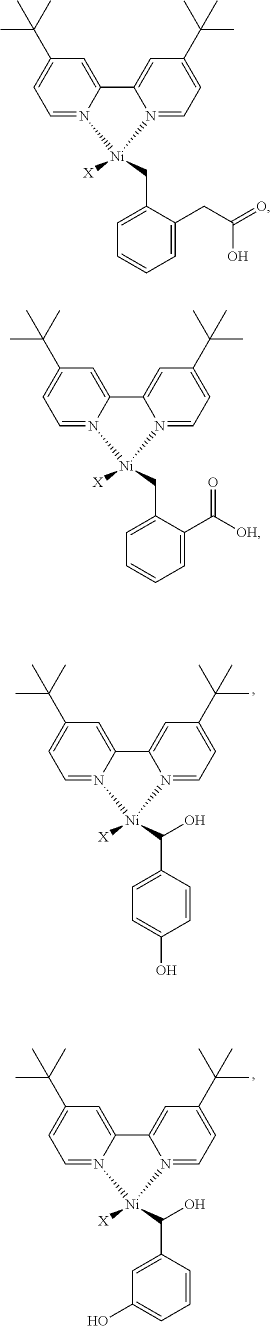

or a combination thereof.

Example 18. The compound of any one of Examples 1-17, wherein X is chlorine.

Example 19. The compound of any one of Examples 1-18 comprising

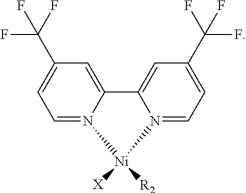

Example 20. The compound of any one of Examples 1-19, wherein R₂ comprises a linear ether.

Example 21. The compound of any one of Examples 1-20, comprising at least one of

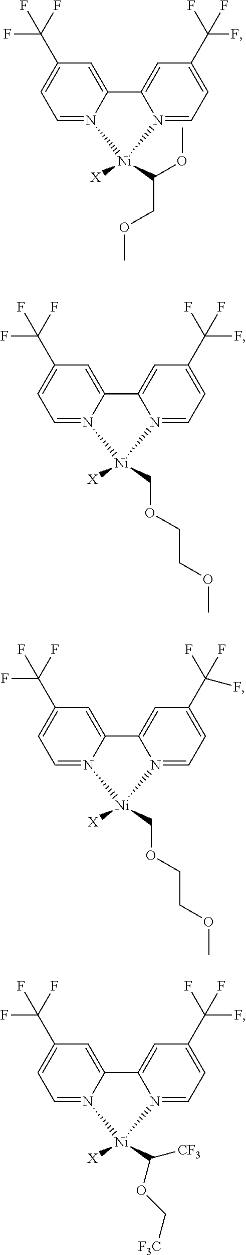

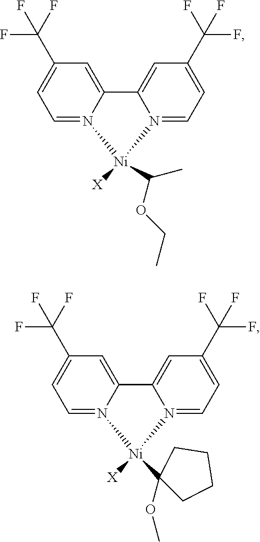

or a combination thereof.

Example 22. The compound of any one of Examples 1-21, wherein X is chlorine.

Example 23. The compound of any one of Examples 1-22, wherein R₂ comprises a cyclic ether.

Example 24. The compound of any one of Examples 1-23, comprising at least one of

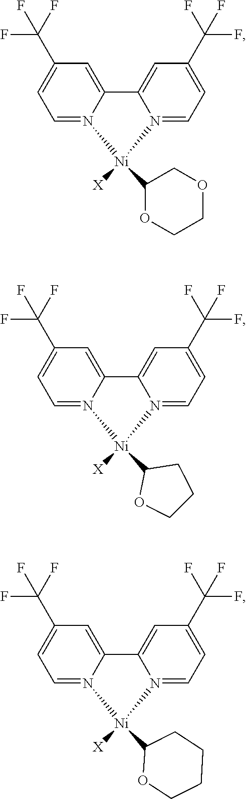

or a combination thereof.

Example 25. The compound of any one of Examples 1-24, wherein X is chlorine.

Example 26. The compound of any one of Examples 1-25, wherein R₂ comprises a benzyl group.

Example 27. The compound of any one of Examples 1-26, comprising at least one of

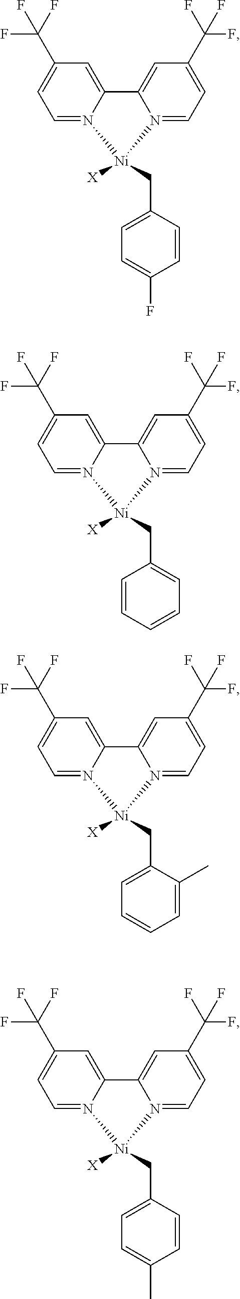

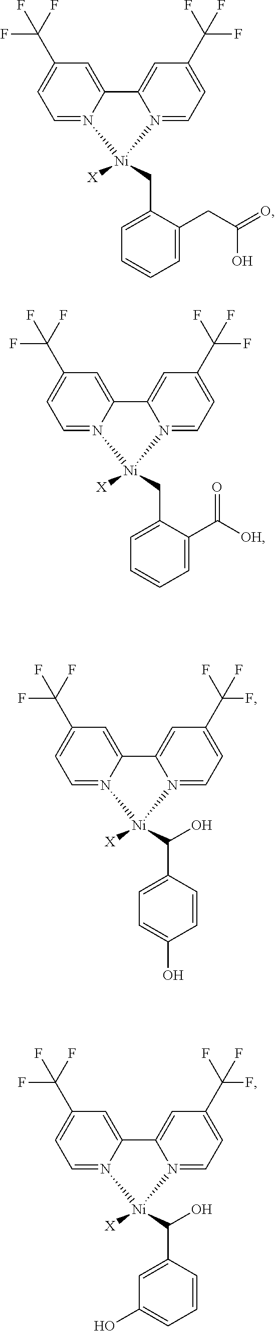

or a combination thereof.

Example 28. The compound of any one of Examples 1-27, wherein X is chlorine.

Example 29. The compound of any one of Examples 1-28 comprising

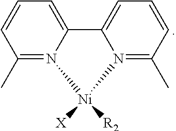

Example 30. The compound of any one of Examples 1-29, wherein R₂ comprises a linear ether.

Example 31. The compound of any one of Examples 1-30, comprising at least one of

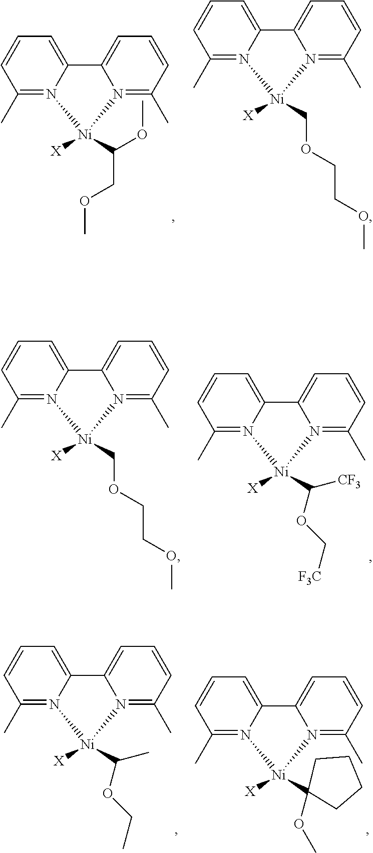

or a combination thereof.

Example 32. The compound of any one of Examples 1-31, wherein X is chlorine.

Example 33. The compound of any one of Examples 1-32, wherein R₂ comprises a cyclic ether.

Example 34. The compound of any one of Examples 1-33, comprising at least one of

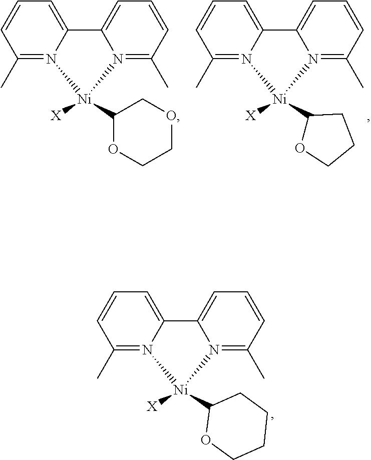

or a combination thereof.

Example 35. The compound of any one of Examples 1-34, wherein X is chlorine.

Example 36. The compound of any one of Examples 1-35, wherein R₂ comprises a benzyl group.

Example 37. The compound of any one of Examples 1-36, comprising at least one of

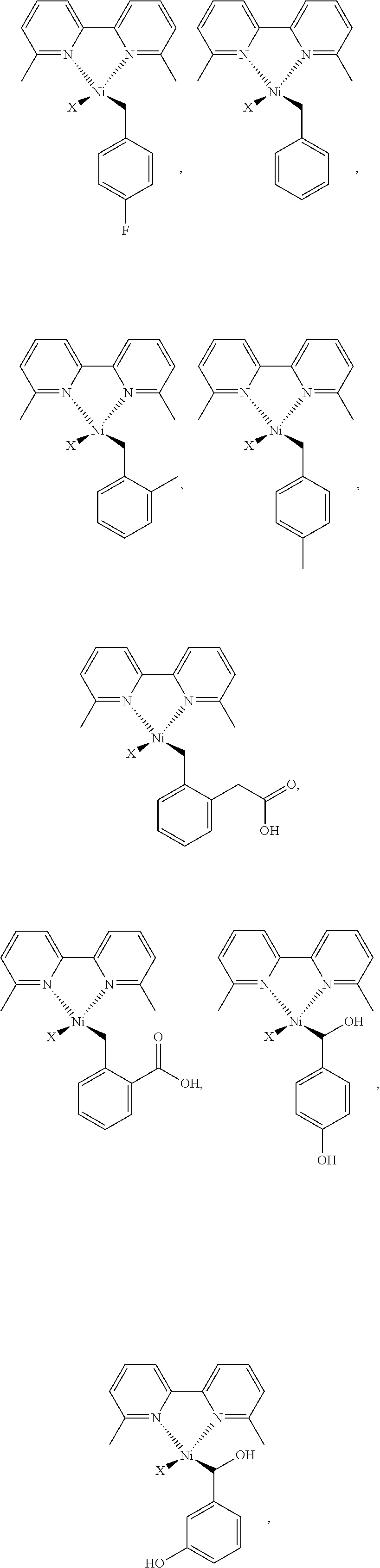

or a combination thereof.

Example 38. The compound of any one of Examples 1-37, wherein X is chlorine.

Example 39. A method comprising: reacting CO₂ and a nickel pre-catalyst with a reactant comprising at least one of a benzyl group, an ether, or a combination thereof to form a carboxylic acid, wherein: the nickel pre-catalyst comprises:

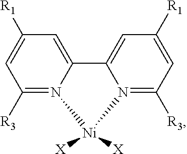

R₁ comprises at least one of a methyl group, a halogen, or a combination thereof, R₃ comprises at least one of hydrogen, a methyl group, or a combination thereof, and X comprises a halogen.

Example 40. The method of Example 39, wherein the reacting further comprises irradiating the nickel pre-catalyst with light having a wavelength between 300 nm and 800 nm or between 350 nm and 500 nm.

Example 41. The method of either Example 39 or Example 40, wherein the reacting further comprises a photosensitizer.

Example 42. The method of any one of Examples 39-41, wherein the photosensitizer comprises a cyanoarene.

Example 43. The method of any one of Examples 39-42, wherein the cyanoarene comprises at least one of 1,2,3,5-tetrakis(carbazol-9-yl)-4,6-dicyanobenzene, 2,4,6-tri(9H-carbazol-9-yl)-5-chloroisophthalonitrile, 2,4,6-tris (diphenylamino)-3,5-difluorobenzonitrile, 2,4,6-tris(diphenylamino)-5-fluoroisophthalonitrile, or a combination thereof.

Example 44. The method of any one of Examples 39-43, wherein the reacting further comprises a base.

Example 45. The method of any one of Examples 39-44, wherein the base comprises at least one of a carbonate, a phosphate, a tertiary amine, or a combination thereof.

Example 46. The method of any one of Examples 39-45, wherein the carbonate at least one of Cs₂CO₃, K₂CO₃, Na₂CO₃, or a combination thereof.

Example 47. The method of any one of Examples 39-46, wherein the phosphate comprises K₃PO₄.

Example 48. The method of any one of Examples 39-47, wherein the tertiary amine comprises at least one of 2,6-dimethylpyridine, triethylamine, 1,4-diazabicyclo[2.2.2]octane, or a combination thereof.

Example 49. The method of any one of Examples 39-48, wherein the reacting further comprises a sacrificial reductant.

Example 50. The method of any one of Examples 39-49, wherein the sacrificial reductant comprises at least one of diethyl 1,4-dihydro-2,6-dimethyl-3,5-pyridinedicarboxylate (HEH), triethylamine, triethanolamine, or a combination thereof.

Example 51. The method of any one of Examples 39-50, wherein: the reacting further comprises forming an intermediate compound that disassociates to form the carboxylic acid, and the intermediate compound comprises:

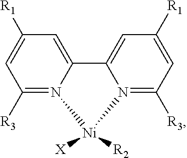

and R₂ comprises the benzyl group, the ether, or a combination thereof.

Example 52. The method of any one of Examples 39-51, wherein the carboxylic acid comprises at least one of

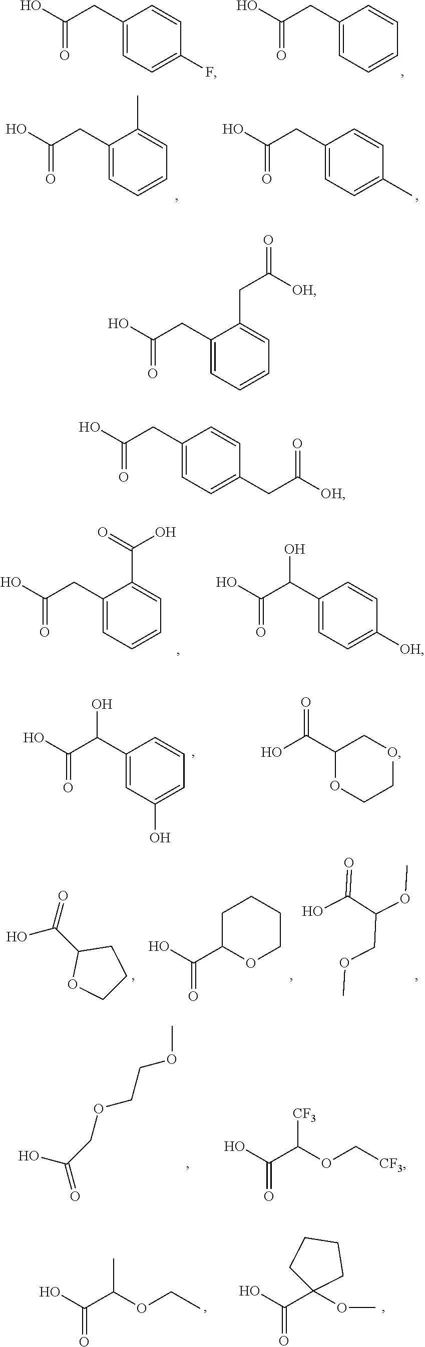

or a combination thereof.

Example 53. The method of any one of Examples 39-52, wherein the reactant comprises at least one of

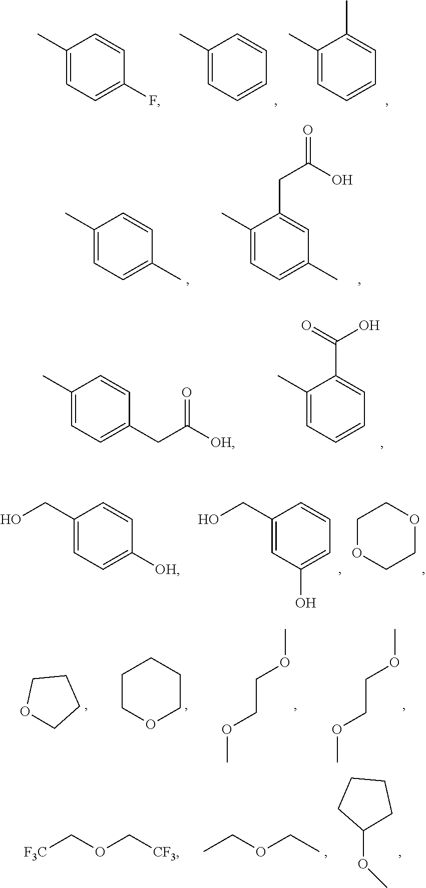

or a combination thereof.

Example 54. The method of any one of Examples 39-53, further comprising, prior to reacting CO₂ and the nickel pre-catalyst with the reactant: reacting a ligand with a nickel complex to form the nickel pre-catalyst, wherein: the ligand comprises

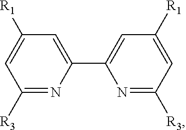

and the nickel complex comprises

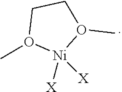

Example 55. The method of any one of Examples 39-54, wherein the reacting is performed using between 380 Torr and 7,600 Torr CO₂ or between 760 Torr and 1,520 Torr CO₂.

Example 56. The method of any one of Examples 39-55, wherein the reacting is performed at a temperature between 0° C. and 200° C. or between 0° C. and 100° C.

Example 57. The method of any one of Examples 39-56, wherein the reacting is performed at a concentration of nickel relative to the reactant between 0.01 mol % and 30 mol % or between 0.05 mol % and 15 mol %.

Example 58. The method of any one of Examples 39-57, wherein the reacting is performed at a concentration of ligand relative to the reactant between 0.001 mol % and 60 mol % or between 0.01 mol % and 30 mol %.

Example 59. The method of any one of Examples 39-58, wherein the reacting is performed at a concentration of photosensitizer relative to the reactant between 0.001 mol % and 60 mol % or between 0.01 mol % and 30 mol %.

Example 60. The method of any one of Examples 39-59, wherein the reacting is performed at a concentration of sacrificial reductant between 0.01 eq and 10 eq or between 0.1 eq and 3 eq.

Example 61. The method of any one of Examples 39-60, wherein the reacting is performed at a concentration of base between 0.01 eq and 10 eq or between 0.1 eq and 3 eq.

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. A compound comprising: 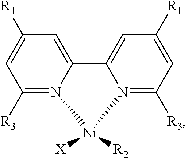
wherein:
R₁ comprises at least one of a methyl group, a halogen, a hydrogen, or a combination thereof,
R₂ comprises at least one of a benzyl group, an ether, or a combination thereof,
R₃ comprises at least one of hydrogen, a methyl group, or a combination thereof, and
X comprises a halogen.

2. The compound of claim 1, further comprising an alkyl linking group positioned between the nickel atom and R₂.

3. The compound of claim 2, wherein the alkyl group is a —CH₂-group.

4. The compound of claim 1, wherein Ri comprises at least one of —C(CH₃)₃, —CF₃, H, or a combination thereof.

5. The compound of claim 1, wherein X comprises at least one iodine, chlorine, bromine, or a combination thereof.

6. The compound of claim 1, wherein the benzyl group is selected from the group consisting of 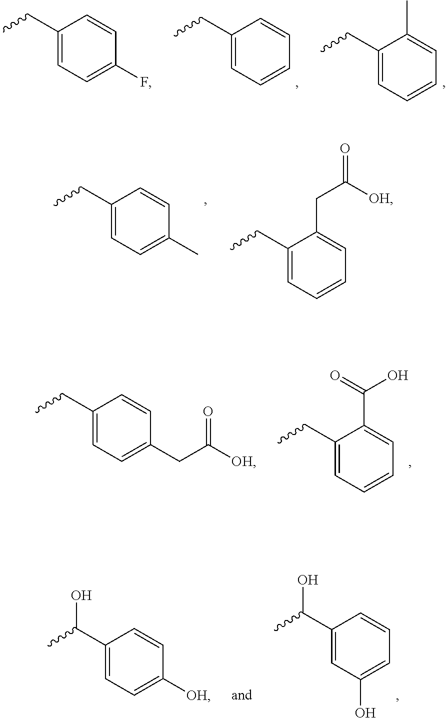
and [symbol] is a covalent bond between R₂ and Ni.

7. The compound of claim 1, wherein the ether is a linear molecule.

8. The compound of claim 7, wherein the linear molecule is selected from the group consisting of 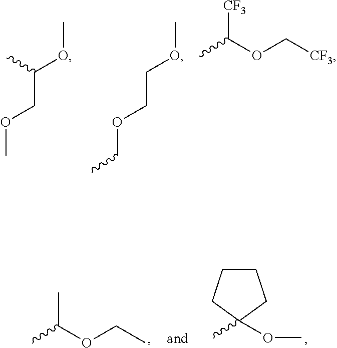
and [symbol] is a covalent bond between R₂ and Ni.

9. The compound of claim 1, wherein the ether is a cyclic molecule.

10. The compound of claim 9, wherein the cyclic molecule is selected from the group consisting of 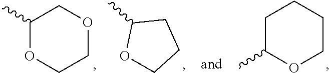
and [symbol] M is a covalent bond between R₂ and Ni.

12. A method comprising:
reacting CO₂ and a nickel pre-catalyst with a reactant comprising at least one of a benzyl group, an ether, or a combination thereof to form a carboxylic acid, wherein:
the nickel pre-catalyst comprises: 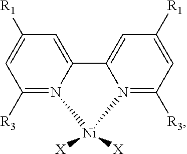
R₁ comprises at least one of a methyl group, a halogen, or a combination thereof,
R₃ comprises at least one of hydrogen, a methyl group, or a combination thereof, and
X comprises a halogen.

13. The method of claim 12, wherein the reacting further comprises irradiating the nickel pre-catalyst with light having a wavelength between 300 nm and 800 nm or between 350 nm and 500 nm.

14. The method of claim 12, wherein the reacting further comprises a photosensitizer.

15. The method of claim 12, wherein the reacting further comprises a base.

16. The method of claim 12, wherein the reacting further comprises a sacrificial reductant.

17. The method of claim 12, wherein:
the reacting further comprises forming an intermediate compound that reacts to form the carboxylic acid, and
the intermediate compound comprises: 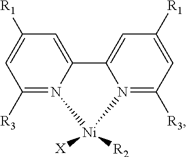
and
R₂ comprises at least one of the benzyl group, the ether, or a combination thereof.

18. The method of claim 12, wherein the carboxylic acid comprises at least one of 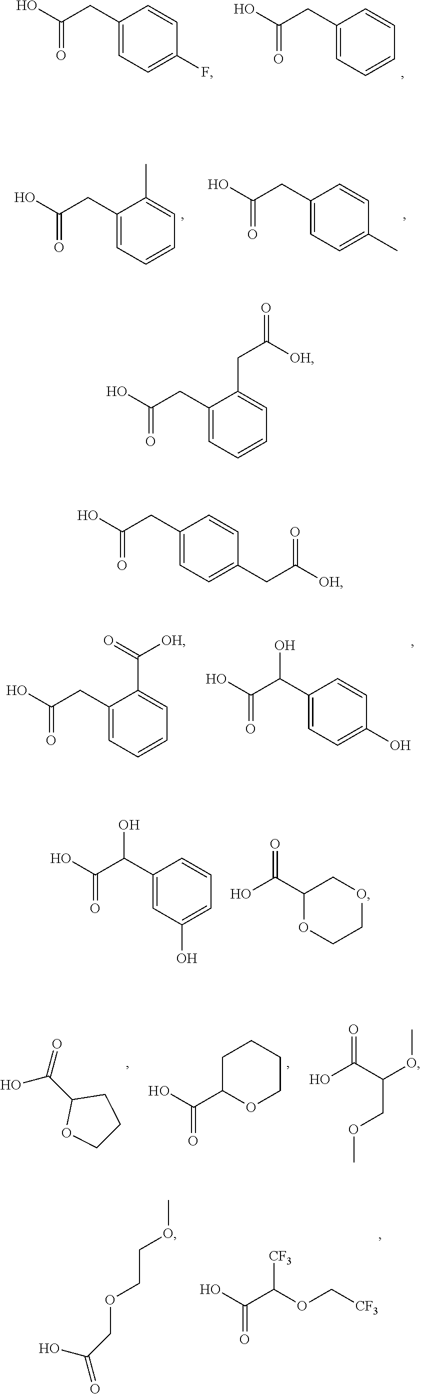 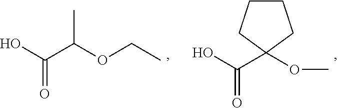
or a combination thereof.

19. The method of claim 12, wherein the reactant comprises at least one of 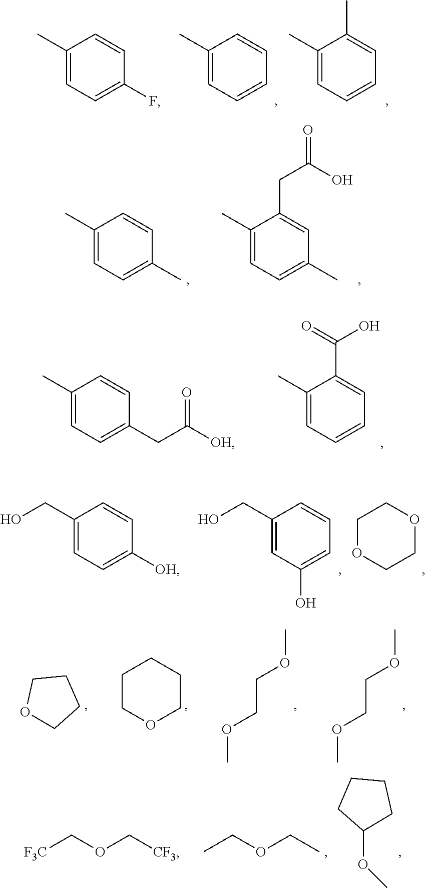
or a combination thereof.

20. The method of claim 17, further comprising, prior to reacting CO₂ and the nickel pre-catalyst with the reactant:
reacting a ligand with a nickel complex to form the nickel pre-catalyst, wherein:
the ligand comprises 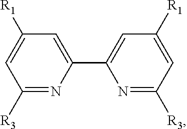
and the nickel complex comprises [chemical structure]
